(19) United States
(12) Patent Application Publication
(10) Pub. No.: US 2026/0198813 A1
(43) Pub. Date: Jul. 16, 2026

(54) ORAL DEVICES WITH HYDROGELS FOR CAPTURE AND DETECTION OF DISEASE BIOMARKERS

(71) Applicant: Align Technology, Inc., San Jose, CA (US)

(72) Inventors: Julie Coletti, Phoenix, AZ (US); Michael Austin Brown, Orlando, FL (US); Mitra Derakhshan, Herndon, VA (US); Allison Warren, Chandler, AZ (US)

(21) Appl. No.: 19/336,211

(22) Filed: Sep. 22, 2025

Related U.S. Application Data
(60) Provisional application No. 63/745,486, filed on Jan. 15, 2025.

(51) Int. Cl. A61B 5/1459; A61B 5/00; A61K 49/00

(52) U.S. Cl. CPC A61B 5/1459; A61B 5/0088; A61B 5/4277; A61B 5/6847; A61K 49/0017

(57) ABSTRACT

Systems and methods for detecting a disease biomarker in a subject are provided. In some embodiments, a system includes: an oral device configured to be positioned within a subject's mouth; a hydrogel carried by the oral device, where the hydrogel includes a polymer and a capture moiety coupled to the polymer, and where the capture moiety is configured to bind to a disease biomarker present in the subject's mouth; and a fluorescent moiety configured to label the disease biomarker bound by the capture moiety.

### CROSS-REFERENCE TO RELATED APPLICATION(S)

The present application claims the benefit of priority to U.S. Provisional Patent Application No. 63/745,486, filed Jan. 15, 2025, which is incorporated by reference herein in its entirety.

### TECHNICAL FIELD

The present technology generally relates to medical devices, and in particular, to oral devices with hydrogels for detection of disease biomarkers.

### BACKGROUND

The lifetime risk for oral cavity and oropharyngeal cancer is about 1 in 59 for men and 1 in 139 for women. The risk is increased for individuals who use tobacco or alcohol, or who have been infected with human papillomavirus (HPV). Conventional approaches for diagnosis of oral cavity and oropharyngeal cancer generally rely upon visual examination by a clinician to identify suspicious lesions, followed by biopsy to confirm the cancer diagnosis. However, visual examination may miss early stage cancers, particularly if the lesions are small or otherwise not immediately apparent, while biopsies may be invasive and inconvenient for the patient. Moreover, visual examination can be highly subjective and may not provide any mechanism for the clinician to systematically track the development of suspicious lesions over time.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIGS. 1A and 1B are perspective and cross-sectional views of a dental appliance including a hydrogel for detection of oral disease biomarkers, in accordance with embodiments of the present technology.

FIGS. 2A-2E are partially schematic illustrations of hydrogel configurations for dental appliances, in accordance with embodiments of the present technology.

FIG. 3A illustrates a lollipop-type oral device including a hydrogel, in accordance with embodiments of the present technology.

FIG. 3B illustrates a lollipop-type oral device including a hydrogel, in accordance with embodiments of the present technology.

FIG. 4 is a flow diagram illustrating a method for detecting an oral disease biomarker in a subject, in accordance with embodiments of the present technology

FIGS. 5A-5C are schematic illustrations of a system for detecting an oral disease biomarker in a subject, in accordance with embodiments of the present technology.

FIGS. 6A-6D are photographs illustrating representative examples of precancerous lesions in the mouth, in accordance with embodiments of the present technology.

FIG. 7 is a flow diagram illustrating a method for detecting an oral disease biomarker in a subject, in accordance with embodiments of the present technology.

FIGS. 8A-8C are schematic illustrations of a system for detecting an oral disease biomarker in a subject, in accordance with embodiments of the present technology.

FIG. 9 is a flow diagram illustrating a method for detecting an oral disease biomarker in a subject, in accordance with embodiments of the present technology.

FIGS. 10A and 10B are schematic illustrations of a system for detecting an oral disease biomarker in a subject, in accordance with embodiments of the present technology.

FIG. 11 illustrates a digital dental workflow for monitoring and/or treating a subject's teeth, in accordance with embodiments of the present technology.

FIG. 12A illustrates a representative example of a tooth repositioning appliance configured in accordance with embodiments of the present technology.

FIG. 12B illustrates a tooth repositioning system including a plurality of appliances, in accordance with embodiments of the present technology.

FIG. 12C illustrates a method of orthodontic treatment using a plurality of appliances, in accordance with embodiments of the present technology.

FIG. 13 illustrates a method for designing an orthodontic appliance, in accordance with embodiments of the present technology.

FIG. 14 illustrates a method for digitally planning an orthodontic treatment and/or design or fabrication of an appliance, in accordance with embodiments of the present technology.

FIG. 15A schematically illustrates a system for performing intraoral scanning and/or generating 3D digital representations of a patient's intraoral cavity, in accordance with embodiments of the present technology.

FIG. 15B is a partially schematic illustration of an example scanner that may be used in the system of FIG. 15A, in accordance with embodiments of the present technology.

### DETAILED DESCRIPTION

The present technology relates to systems, devices, and methods for detection of diseases and conditions of the oral cavity, such as oral cavity and oropharyngeal cancer (collectively referred to herein as “oral cancer”). In some embodiments, for example, a system for detecting a disease biomarker in a subject includes an oral device configured to be positioned within a subject's mouth (e.g., a dental appliance) and a hydrogel carried by the oral device. The hydrogel can be used to detect disease biomarkers that are present in the subject's mouth. For instance, the disease biomarker can be indicative of one or more of the following conditions: oral cancer, caries, orthodontic bone remodeling, periodontitis, Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse (e.g., tobacco abuse, alcohol abuse).

In some embodiments, the hydrogel includes a polymer and a capture moiety coupled to the polymer, where the capture moiety is configured to bind to a disease biomarker present in the subject's mouth. The system can further include a fluorescent moiety configured to label the disease biomarker bound by the capture moiety. The system can also include an imaging device (e.g., an intraoral or extraoral scanner, a mobile device) that obtains image data of the hydrogel, where the presence of the labeled disease biomarker in the image data is used to diagnose the subject with a disease or condition.

In some embodiments, the hydrogel includes a polymer and a fluorescent moiety. When the oral device is positioned within the subject's mouth, the hydrogel can release the fluorescent moiety into the subject's mouth, and the fluorescent moiety can label a disease biomarker present in the subject's mouth. The system can also include an imaging device (e.g., an intraoral scanner, a mobile device) that obtains image data of the subject's mouth, where the presence of the labeled disease biomarker in the image data is used to diagnose the subject with a disease or condition.

In some embodiments, the hydrogel includes a polymer and a fluorescent moiety. The fluorescent moiety can be in an inactive state before the oral device is positioned in the subject's mouth. When the oral device is positioned within the subject's mouth, the fluorescent moiety can interact with a disease biomarker in the subject's mouth, and the interaction can cause the fluorescent moiety to be converted into an active state. The system can also include an imaging device (e.g., an intraoral or extraoral scanner, a mobile device) that obtains image data of the hydrogel, where the presence of the active fluorescent moiety in the image data is used to diagnose the subject with a disease or condition.

The present technology can provide numerous advantages compared to conventional approaches for diagnosing oral disease and conditions. For instance, the hydrogels and devices described herein can be used to label disease biomarkers with a high degree of sensitivity, therefore allowing for earlier diagnosis of diseases and improved treatment outcomes. Moreover, the use of an oral device to deliver hydrogels to the mouth is less invasive than incisional biopsy and safer than general tissue staining and evaluation with UV light. Because this approach is more convenient for patients, there can be greater adoption for routine screening of at-risk and low-risk populations alike. Moreover, the present technology can be integrated into digital workflows for dental monitoring and treatment, thus providing a structured solution for diagnosis of oral diseases and conditions that is compatible with existing treatment planning, reimbursement, and billing practices.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Detection of Oral Disease Biomarkers with Hydrogels

The present technology relates to oral devices with hydrogels for detection of biomarkers associated with diseases or conditions of the oral cavity and/or biomarkers for disease or conditions of other anatomical locations that may be detected in the oral cavity (referred to herein as “oral disease biomarkers”). In some embodiments, an oral device of the present technology includes a hydrogel and at least one detection agent carried by the hydrogel. The detection agent can interact with the oral disease biomarkers to capture and/or label the biomarkers for detection, e.g., via fluorescent imaging and/or other imaging modalities.

### A. Hydrogels

The hydrogels of the present technology can include a network of polymers that are crosslinked with each other through covalent bonds and/or through non-covalent interactions (e.g., ionic bonding, hydrogen bonding, hydrophobic interactions, Van der Waals interactions). The hydrogel may be hydrophilic due to the presence of hydrophilic functional groups on the polymers (e.g., amino, carboxyl, amide, and/or hydroxyl groups), and can therefore absorb water and/or physiological fluids (e.g., saliva, gingival crevicular fluid (GCF)) when placed in the mouth. Thus, oral disease biomarkers that are present in the physiological fluids in the mouth can infiltrate into the hydrogel via diffusion and interact with (e.g., be captured and/or labeled by) the detection agent(s) carried by the hydrogel. Alternatively or in combination, detection agent(s) that are not immobilized in the hydrogel can be released from the hydrogel via diffusion and interact with (e.g., capture and/or label) oral disease biomarkers in the mouth.

The hydrogel can be composed of one or more polymers, such as a single type of polymer or a combination of multiple different polymers (e.g., two, three, four, five, or more different polymers). The polymers can be naturally occurring polymers (e.g., polysaccharides, polypeptides), synthetic polymers, or combinations thereof. Examples of polymers that may be used in the hydrogels described herein include agarose, alginate, cellulose (e.g., cellulose derivatives such as hydroxypropylmethylcellulose (HPMC), hydroxyethyl cellulose (HEC), hydroxypropylcellulose (HPC), ethylcellulose (EC), methylcellulose (MC), hydroxyethylmethylcellulose (HEMC), carboxymethylcellulose (CMC), carboxymethyl ethyl cellulose (CMEC)), chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, and polyvinylpyrrolidone, as well as derivatives and combinations (e.g., mixtures, copolymers) thereof.

In some embodiments, the hydrogels described herein are biocompatible. A biocompatible material can be a material that is, along with any metabolites or degradation products thereof, generally non-toxic to the subject, and does not cause any significant adverse effects to the subject, at concentrations resulting from the degradation of the administered materials. A biocompatible material can be a material that does not elicit a significant inflammatory or immune response when administered to a subject.

In some embodiments, the hydrogels described herein are biodegradable. A biodegradable material can be a material that will degrade or erode under physiological conditions to smaller units or chemical species that are capable of being metabolized, eliminated, or excreted by the subject. In other embodiments, however, the hydrogels described herein may be non-biodegradable.

In some embodiments, the hydrogels described herein are “smart” hydrogels that are configured to respond to an external stimulus, such as pH, temperature, light, magnetic fields, electrical fields, humidity, ionic strength, force, biological signals (e.g., enzymes), etc. The response can include, for example, a change in size (e.g., swelling, shrinking), a change in shape (e.g., deforming, bending, twisting, folding), a change in mechanical properties (e.g., stiffness), a change in optical properties (e.g., color, translucency), a change in diffusivity, release of an encapsulated agent, or combinations thereof. In some embodiments, the response is reversible, in that the hydrogel can revert back to its original state when the stimulus is removed. In other embodiments, the response may be irreversible such that the hydrogel remains in the new state even after removal of the stimulus.

Smart hydrogels can be composed of one or more stimuli-responsive polymers. For example, thermosensitive polymers such as poly(N-isopropylacrylamide), poly(N-vinylcaprolactam), poly(ethylene oxide)-poly(propylene oxide), and poloxamers can respond to physiological temperature changes with variations in physical properties including shape deformations, shrinking-swelling behavior, and stiffening. In some embodiments, the stimuli-responsive polymer can include functional groups that undergo chemical reactions in response to stimuli, such as photocleavable groups, thermally-cleavable groups, enzymatically-cleavable groups, etc. In some embodiments, smart hydrogels are used to release an encapsulated detection agent only upon exposure to a desired stimulus, such as exposure to physiological temperature, humidity, and/or ionic strength.

### B. Detection Agents

The hydrogels herein can be used with one or more detection agents that allow for detection of oral disease biomarkers. For example, the hydrogel can encapsulate at least one detection agent, such as a capture moiety that binds to oral disease biomarkers, a fluorescent moiety that labels oral disease biomarkers for detection, or a combination thereof (e.g., a single molecule that both binds to and labels the biomarkers). Encapsulation of detection agents in a hydrogel can be accomplished through various methods, including covalent conjugation and/or non-covalent interactions (e.g., ionic bonding, hydrogen bonding, hydrophobic interactions, Van der Waals interactions). In some embodiments, the detection agent is immobilized within the hydrogel, e.g., via covalent bonding to polymers of the hydrogel and/or physical entrapment within the hydrogel (e.g., if the size of the detection agent is larger than the mesh size of the polymer network). In other embodiments, the detection agent may not be immobilized within the hydrogel and thus may be capable of migrating out of the hydrogel, e.g., via diffusion, in response to stimuli, etc.

Optionally, one or more detection agents can be provided separately from the hydrogel and applied to the hydrogel after the hydrogel has been used to capture oral disease biomarkers present in the mouth, such as a fluorescent moiety for labeling the captured biomarkers. A detection agent may be applied to the hydrogel in any suitable manner, such as immersing the hydrogel in a solution of the detection agent; spraying, dipping, dripping, or brushing a solution of the detection agent onto a portion of or the entire hydrogel; etc.

In some embodiments, the hydrogels herein include a capture moiety that binds to an oral disease biomarker, such as an antibody (e.g., a full-length antibody, an antibody fragment such as an Fab or scFv fragment), an aptamer, a peptide, a polysaccharide, or a small molecule. The capture moiety may be immobilized within the hydrogel to allow the hydrogel to capture oral disease biomarkers that enter the hydrogel when the hydrogel is placed in the subject's mouth. The binding of the capture moiety to the oral disease biomarker may be reversible or irreversible. In some embodiments, the capture moiety specifically binds to the oral disease biomarker and exhibits little or no binding affinity for other species present in the mouth (e.g., salivary proteins). In other embodiments, the capture moiety also binds nonspecifically to other species present in the mouth, in which case another detection agent may be used to distinguish the oral disease biomarker from the other species, such as a fluorescent moiety that binds specifically to the oral disease biomarker.

In some embodiments, the hydrogels herein include or are otherwise used with a fluorescent moiety that is configured to label an oral disease biomarker for detection. The fluorescent moiety may be immobilized within the hydrogel to allow the hydrogel to label oral disease biomarkers that enter the hydrogel when the hydrogel is placed in the subject's mouth. Alternatively, the fluorescent moiety may be released from the hydrogel to label oral disease biomarkers in situ within the subject's mouth. Optionally, the fluorescent moiety may not be present when the hydrogel is placed in the subject's mouth and may be applied to the hydrogel in a later step (e.g., to label oral disease biomarkers captured by the hydrogel).

The fluorescent moiety can include (1) a component that binds to the oral disease biomarker, such as an antibody (e.g., a full-length antibody, an antibody fragment such as an Fab or scFv fragment), an aptamer, a peptide, a polysaccharide, or a small molecule, and (2) a fluorescent molecule, such as an organic dye, a polymer dye, a fluorescent protein, or a quantum dot. The binding of the fluorescent moiety to the oral disease biomarker may be reversible or irreversible. In some embodiments, the fluorescent moiety specifically binds to the oral disease biomarker and exhibits little or no binding affinity for other species present in the mouth (e.g., salivary proteins). In other embodiments, the fluorescent moiety also binds nonspecifically to other species present in the mouth, in which case another detection agent may be used to distinguish the oral disease biomarker from the other species, such as a capture moiety that binds specifically to the oral disease biomarker.

The fluorescent molecule can have any suitable excitation wavelength and emission wavelength. For instance, the excitation wavelength can be within a range from 300 nm to 350 nm, 350 nm to 400 nm, 400 nm to 450 nm, 450 nm to 500 nm, 500 nm to 550 nm, 550 nm to 600 nm, 600 nm to 650 nm, 650 nm to 700 nm, 700 nm to 750 nm, or 750 nm to 800 nm. The emission wavelength can be in the ultraviolet range, the visible range, the near-infrared (NIR) range, or the infrared range, such as within a range from 300 nm to 350 nm, 350 nm to 400 nm, 400 nm to 450 nm, 450 nm to 500 nm, 500 nm to 550 nm, 550 nm to 600 nm, 600 nm to 650 nm, 650 nm to 700 nm, 700 nm to 750 nm, or 750 nm to 800 nm.

In some embodiments, the fluorescent molecule is a fluorescent dye, such as an organic dye or a polymer dye. Examples of fluorescent dyes include coumarin and coumarin derivatives, cyanine and cyanine derivatives (e.g., Cy2, Cy3, Cy5, Cy5.5, Cy7), fluorescein and fluorescein derivatives (e.g., fluorescein isothiocyanate (FITC)), and rhodamine and rhodamine derivatives (e.g., tetramethylrhodamine (TRITC), Texas Red).

In some embodiments, the fluorescent molecule is a fluorescent protein. Examples of fluorescent proteins include blue fluorescent proteins (e.g., Sirius, Azurite, EBFP, EBFP2, mTagBFP), cyan fluorescent proteins (e.g., ECFP, Cerulean, CyPet, SCFP, TagCFP, AmCyan, Midoriishi Cyan, mTFP1), green fluorescent proteins (e.g., EGFP, Emerald, Superfolder avGFP, T-Sapphire, Azami Green, mWasabi, ZsGreen, TagGFP, TagGFP2, TurboGFP, CopGFP, AceGFP), yellow fluorescent proteins (e.g., EYFP, Topaz, Venus, Citrine, YPet, SYFP, mAmetrine, Tag YFP, Turbo YFP, ZsYellow, PhiYFP), orange fluorescent proteins (e.g., Kusabira Orange, Kusabira Orange2, mOrange, mOrange2, dTomato, dTomato-Tandem, DsRed, DsRed2, DsRed-Express (T1), DsRed-Express2, DsRed-Max, DsRed-Monomer, TurboRFP, TagRFP, TagRFP-T), red fluorescent proteins (e.g., mRuby, mApple, mStrawberry, AsRed2, mRFP1, jRed, mCherry, eqFP611, tdRFP611, HcRed1, mRaspberry), and far-red fluorescent proteins (e.g., tdRFP639, mKate, mKate2, Katushka, tdKatushka, HcRed-Tandem, mPlum, AQ143).

In some embodiments, the fluorescent molecule is a quantum dot. Quantum dots are nanocrystals of semiconducting material (e.g., indium phosphide (InP), indium gallium phosphide (InGaP), gallium nitride (GaN), cadmium selenide (CdSe) cadmium telluride (CdTe)) The particle size of a quantum dot is generally within a range from 1 nm to 10 nm. The optical properties of quantum dots may be controlled by the material selection, particle size, particle size distribution, and/or surface chemistry.

In some embodiments, the fluorescent molecule is a “smart” molecule that is converted from an inactive (e.g., non-fluorescent) state to an active (e.g., fluorescent) state in response to a stimulus, such as interaction with (e.g., binding to) an oral disease biomarker. For instance, the fluorescent moiety can be converted into the active state via an enzymatic reaction (e.g., if the oral disease biomarker is an enzyme), a conformational change (e.g., triggered by binding of the oral disease biomarker to the fluorescent moiety), and/or removal of a fluorescence quencher (e.g., via enzymatic cleavage of the quencher from the fluorescent moiety, in embodiments where the oral disease biomarker is an enzyme). In other embodiments, however, the fluorescent molecule may constitutively be in the active fluorescent state such that no conversion process is needed.

Although certain embodiments herein are described in connection with fluorescent moieties for fluorescent labeling of oral disease biomarkers, this is not intended to be limiting. Other types of detectable labels known to those of skill in the art may be used in the present technology, such as colorimetric labels and luminescent labels. Any reference herein to a “fluorescent moiety” may be modified as appropriate to use other types of labeling modalities.

### C. Oral Disease Biomarkers

The hydrogels and detection agents described herein can be used to capture and/or label biomarkers for a wide variety of diseases and conditions. Examples of disease and conditions that may be detected using the systems and devices described herein include oral cancer (e.g., oral cavity cancer, oropharyngeal cancer), caries, orthodontic bone remodeling, periodontitis, Alzheimer's disease, asthma, cardiovascular disease, chronic obstructive pulmonary disease (COPD), cystic fibrosis, diabetes (e.g., Type II diabetes), Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, and substance abuse (e.g., tobacco abuse, alcohol abuse).

Oral disease biomarkers can include proteins, peptides, small molecules, nucleic acids (e.g., DNA, RNA), polysaccharides, and lipids, for example. Oral disease biomarkers may be found in physiological fluids of the subject's mouth (e.g., saliva, GCF) and/or in tissues of the mouth (e.g., buccal mucosa, lips, teeth, gingiva, tongue, hard palate, soft palate, retromolar trigone, tonsil, uvula, floor of the mouth, throat). Oral disease biomarkers may be localized to the site of the disease or condition (e.g., to the location of a cancerous lesion) or may be present systemically.

In some embodiments, the disease or condition is oral cancer, such as a cancer of the lips, tongue, floor of the mouth, palate, gingiva, alveolar mucosa, tonsils, uvula, or salivary glands. The oral cancer may be oral squamous cell carcinoma (OSCC). The oral disease biomarker for the oral cancer may be a tumor antigen, such as a tumor-specific antigen or a tumor-associated antigen. Examples of oral disease biomarkers that may be used to detect oral cancer include DUSP1, H3F3a, OAZ1, S100P, SAT, M2BP, MRP14, PMAIP1, PTPNI, DAPK1, p16INK4a, chemerin, MMP-9, phytosphingosine, pipecolinic acid, actin, IL-1β, IL-6, IL-8, and TNF-α.

In some embodiments, the disease or condition is caries, and the associated oral disease biomarkers are IL-4, IL-13, IL-2RA, and/or eotaxin/CCL11. Monitoring of caries via biomarkers can be used to assess the subject's risk of developing caries and/or to analyze the severity of existing caries.

In some embodiments, the disease or condition is orthodontic bone remodeling, and the associated oral disease biomarkers are IL-1Iβ, IL-6, TNF-α, GM-CSF, M-CSF, IGF-I, IGF-II acid phosphatase, aspartate aminotransferase, alkaline phosphatase, lactate dehydrogenase, collagenase, MMP-1, MMP-2, MMP-8, cathepsin B, TRAP, osteocalcin, osteonectin, osteopontin, dental matrix protein-1, dentin phosphophoryn, dentin sialoprotein, FGF, TGF-, PDGF, BMP-2, integrins, glucose-6-phosphate dehydrogenase, 3H-urinidine, and/or c-fos. Bone remodeling enables orthodontic tooth movement, and thus monitoring of bone remodeling via biomarkers may be incorporated into various aspects of dental treatment planning, such as diagnosis of the subject's condition, determining personalized care (e.g., variances in responsiveness may be factored into the treatment plan), monitoring before and/or during treatment stages, and/or monitoring during the retention stage (e.g., for unwanted movement).

In some embodiments, the disease or condition is periodontitis, and the associated oral disease biomarkers are cathepsin G, elastase, elastase inhibitors, C-reactive protein, IL-1β, IL-6, MMP-8, creatine kinase, lactate dehydrogenase, aspartate aminotransferase, alanine aminotransferase, gamma-glutamyl transferase, acid phosphatase, alkaline phosphatase, PGE2, and/or TNF-α.

In some embodiments, the disease or condition is diabetes (e.g., Type II diabetes), and the associated oral disease biomarkers are glucose and/or lactoferrin. Prediction of periodontitis disease progression and responsiveness to treatment can be challenging from clinical presentation alone; accordingly, monitoring of periodontitis via biomarkers may be used to establish periodontal status (e.g., stable/inactive) before starting and/or during dental treatment. Oral disease biomarkers for periodontitis may also be used for monitoring of peri-implantitis near dental implants.

In some embodiments, the disease or condition is cardiovascular disease (e.g., atherosclerosis, myocardial infarction, stroke), and the associated oral disease biomarkers are troponin, C-reactive protein, myoglobin, myeloperoxidase, α-2-HS-glycoprotein, lactoferrin, and/or galectin-3.

In some embodiments, the disease or condition is Alzheimer's disease, and the associated oral disease biomarkers are Aβ42 and/or trehalose.

In some embodiments, the disease or condition is chronic obstructive pulmonary disease (COPD), and the associated oral disease biomarkers are C-reactive protein (CRP), fibrinogen, IL-6, IL-8 and/or TNF-α.

In some embodiments, the disease or condition is cystic fibrosis, and the associated oral disease biomarkers are epidermal growth factor and/or calcium ions.

In some embodiments, the disease or condition is Epstein-Barr virus (EBV) infection, and the associated oral disease biomarkers are EBV viral RNA and/or anti-EBV antibodies.

In some embodiments, the disease or condition is gastroesophageal reflux disease (GERD), and the associated oral disease biomarker is pepsin.

In some embodiments, the disease or condition is hepatitis, and the associated oral disease biomarkers are hepatitis viral RNA and/or anti-hepatitis antibodies.

In some embodiments, the disease or condition is human immunodeficiency virus (HIV) infection, and the associated oral disease biomarkers are HIV viral RNA and/or anti-HIV antibodies.

In some embodiments, the disease or condition is human papillomavirus (HPV) infection, and the associated oral disease biomarkers are HPV viral RNA and/or anti-HPV antibodies.

In some embodiments, the disease or condition is Huntington's disease, and the associated oral disease biomarkers are Huntingtin protein (HTT) and/or mutant Hungtingtin protein (mHTT).

In some embodiments, the disease or condition is oral candidiasis, and the associated oral disease biomarkers are mannan, (1,3)-β-D-glucan (BDG), Candida albicans germ tube antibodies (CAGTA), and/or Candida DNA.

In some embodiments, the disease or condition is osteoporosis, and the associated oral disease biomarkers are LRP5, ATF4, integrins, TGF β-I, TGF β-II, IGF-I, IGF-II, PDGF, FGF, glucose-6-phosphate dehydrogenase, 3H-urinidine, c-fos, TRAP, TNF-α, M-CSF, GM-CSF, PGE2, OPG, and/or RANKL.

In some embodiments, the disease or condition is Parkinson's disease, and the associated oral disease biomarkers are SNCA, LRRK2, DJ-1, PRKN, PINK1, and/or GBA.

In some embodiments, the disease or condition is sickle cell anemia, and the associated oral disease biomarkers are fetal hemoglobin, a-thalassemia, CRP, spLA2, IL-2 IL-3, IL-6, IL-8, IL-10, urinary cysteinyl leukotriene E4, prostaglandin-E2, CA 15-3, soluble CD40 ligand, HSP-70, ferritin, angiopoietin 1, angiopoietin 2, stromal derived factor 1, TNF-α, TNF receptor-1, LDH-1, triglycerides, apolipoprotein A-1, VEGF, PGF, and/or ET-1.

In some embodiments, the disease or condition is Sjogren's syndrome, and the associated oral disease biomarker is salivary β-2 microglobulin.

In some embodiments, the disease or condition is substance abuse (e.g., tobacco abuse, alcohol abuse). For example, oral disease biomarkers for alcohol abuse can include acetaldehyde, ethyl glucuronide, fatty acid ethyl esters, and/or sialic acid.

### D. Devices, Systems and Methods for Detection of Oral Disease Biomarkers

The hydrogels of the present technology can be carried by or otherwise incorporated into an oral device. The oral device can be any device that is configured to be inserted partially or entirely into the subject's mouth, such that the hydrogel carried by the oral device is exposed to oral physiologic fluids (e.g., saliva, GCF) and thus can be infiltrated by oral disease biomarkers present in the mouth and/or can release detection agents for the oral disease biomarkers into the mouth.

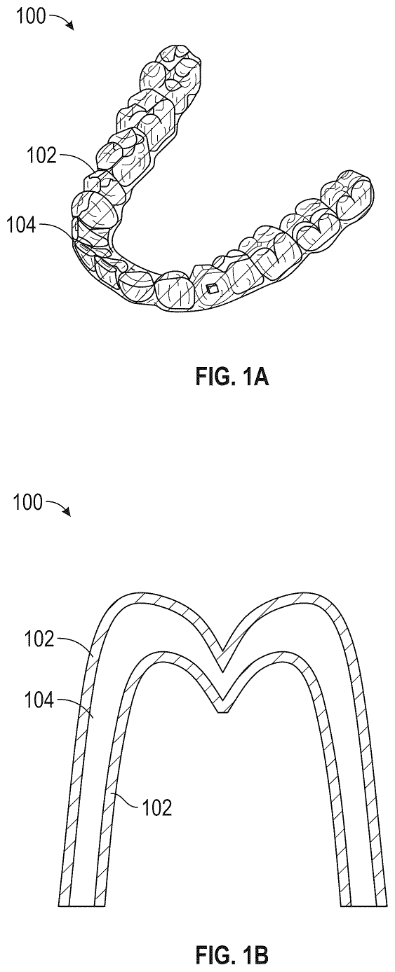

FIGS. 1A and 1B are perspective and cross-sectional views of a dental appliance 100 including a hydrogel 102 for detection of oral disease biomarkers, in accordance with embodiments of the present technology. The dental appliance 100 can be an aligner, retainer, palatal expander, mouthguard, nightguard, oral sleep apnea device, attachment placement template, splint, etc. In the illustrated embodiment, the dental appliance 100 includes a shell 104 having a plurality of cavities for receiving some or all of the teeth of a subject's dental arch. The shell 104 can be a polymeric shell that is fabricated via thermoforming or additive manufacturing, for example.

In some embodiments, the dental appliance 100 is an aligner that is configured to apply forces to one or more teeth to reposition teeth in accordance with a dental treatment plan. The aligner can be one of a series of aligners that incrementally reposition the teeth from an initial arrangement toward a target arrangement via a series of treatment stages of the dental treatment plan, with each aligner being configured to implement a respective treatment stage (e.g., reposition the teeth from a current arrangement toward an intermediate arrangement specified by the treatment stage).

As another example, the dental appliance 100 can be a palatal expander that is configured to apply forces to one or more teeth to expand the subject's palate in accordance with a dental treatment plan. The palatal expander can be one of a series of palatal expanders that incrementally expand the palate from an initial width toward a target width via a series of treatment stages of the dental treatment plan, with each palatal expander being configured to implement a respective treatment stage (e.g., expand the palate from a current width toward an intermediate width specified by the treatment stage).

In other embodiments, however, the dental appliance 100 is not intended to apply treatment forces to the teeth. For instance, the appliance 100 can be a retainer that maintains teeth in a current arrangement (e.g., as part of a holding phase of a dental treatment plan), a mouthguard or nightguard that receives and protects teeth in a current arrangement, etc.

Optionally, the dental appliance 100 may be a specialized hydrogel delivery appliance. Such an appliance can conform to the subject's dentition and can include pockets (e.g., formed in the shell 104) that hold the hydrogel 102 for delivery to various areas of the dentition. The hydrogel delivery appliance may be fabricated (e.g., additively manufactured, thermoformed) during the initial scan of the subject's teeth and/or can conform to the initial (e.g., pre-treatment) arrangement of the teeth. Alternatively, the hydrogel delivery appliance can be used during an intermediate treatment stage and/or can conform to the subject's dentition in conjunction with an intended visit/check-up or a specific treatment stage where progress tracking is of interest.

Some or all of the treatment stages of a dental treatment plan may use a dental appliance 100 with a hydrogel 102. For example, the dental appliance 100 can be used during a treatment stage where dental monitoring and/or progress tracking is intended to be performed, e.g., to assess whether the subject's teeth are progressing as planned, as well as to identify whether the subject has or is at risk of developing an oral disease or condition. In some embodiments, the dental appliance 100 can be used for a stage of the dental treatment plan that is specifically intended for monitoring for the oral disease or condition, in which case the dental appliance 100 can be packaged with the hydrogel 102 and delivered to the subject (e.g., via mail or at a special visit to the dentist.

Additional features and examples of dental appliances and associated methods that are applicable to the dental appliance 100 are discussed in Section II below.

The hydrogel 102 can be any of the embodiments described herein, e.g., in Section I.A above. For instance, the hydrogel 102 can include one or more detection agents, such as capture moieties and/or fluorescent moieties (e.g., as discussed in Section I.B above), to detect one or more oral disease biomarkers that may be present in the subject's mouth (e.g., as discussed in Section I.C above). The hydrogel 102 can be provided in any suitable form factor, such as a coating, layer, film, membrane, patch, capsule, button, etc. The hydrogel 102 may have a uniform thickness or may have a variable thickness. The thickness of the hydrogel 102 can be within a range from 10 μm to 10 mm, 10 μm to 1 mm, 10 μm to 100 μm, 10 μm to 50 μm, 50 μm to 10 mm, 50 μm to 1 mm, 50 μm to 100 μm, 100 μm to 10 mm, 100 μm to 1 mm, or 1 mm to 10 mm. The hydrogel 102 can be coupled to the dental appliance 100 via adhesives, bonding, fasteners, coating (e.g., dip coating, spray coating, spin coating), or suitable combinations thereof.

The hydrogel 102 can be located at any portion of the dental appliance 100 that will come into contact with physiological fluids when the appliance 100 is placed in the subject's mouth. In the illustrated embodiment, the hydrogel 102 covers all of the exposed surfaces of the shell 104, including both the external surface (e.g., the surface facing away from the received teeth) and the internal surface (the surface facing toward the received teeth), and spanning the buccal, lingual, and occlusal surfaces of the shell 104. In other embodiments, however, the hydrogel 102 can be configured differently, e.g., the hydrogel 102 can be located on the external surface only, the internal surface only, on the occlusal surface only, the lingual surface only, the buccal surface only, the gingival edge only, or suitable combinations thereof (e.g., external buccal and lingual surfaces only). Moreover, the hydrogel 102 may cover all of the teeth receiving cavities of the shell 104 (e.g., as shown in FIG. 1A) or may cover only some of the teeth receiving cavities of the shell 104 (e.g., the anterior teeth receiving cavities only, the posterior teeth receiving cavities only, an individual tooth receiving cavity only).

The location of the hydrogel 102 may be selected based on the expected locations of oral disease biomarkers of interest. For instance, if the hydrogel 102 is used to detect an oral disease biomarker that is expected to be present on the gingiva, the hydrogel 102 can be located on a portion of the appliance 100 that is adjacent or proximate to the gingiva (e.g., the gingival edges of the shell 104). As another example, if the hydrogel 102 is used to detect an oral disease marker that is expected to be present on the tongue and/or the floor of the mouth, the hydrogel 102 can be located on a portion of the appliance 100 that is adjacent or proximate to the tongue and/or the floor of the mouth (e.g., the lingual surfaces of the shell 104). In a further example, if the hydrogel 102 is used to detect an oral disease marker that is expected to be present on the palate, the hydrogel 102 can be located on a portion of the dental appliance 100 that is adjacent or proximate to the palate (e.g., a palatal portion—not shown in FIG. 1A).

Optionally, the shell 104 can include additional structures that are specifically designed for carrying the hydrogel 102, such as extensions, projections, flaps, arms, receptacles, pockets, etc., and the hydrogel 102 can be coated onto and/or disposed within the additional structures. The additional structures can be located at any suitable portion of the shell 104 (e.g., that does not interfere with the dental treatment and/or cause discomfort), such as the external surface, internal surface, occlusal surface, lingual surface, buccal surface, gingival edge, palatal portion, etc.

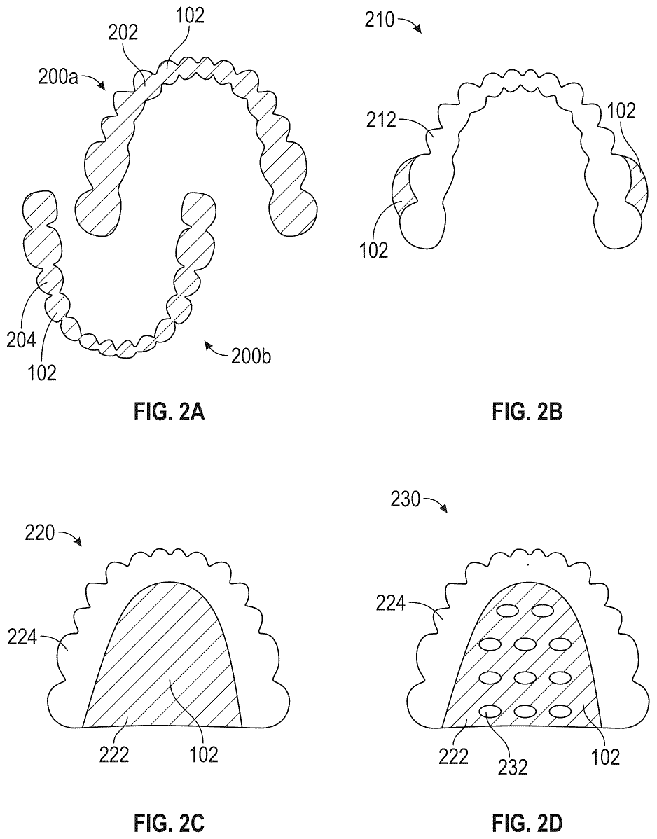

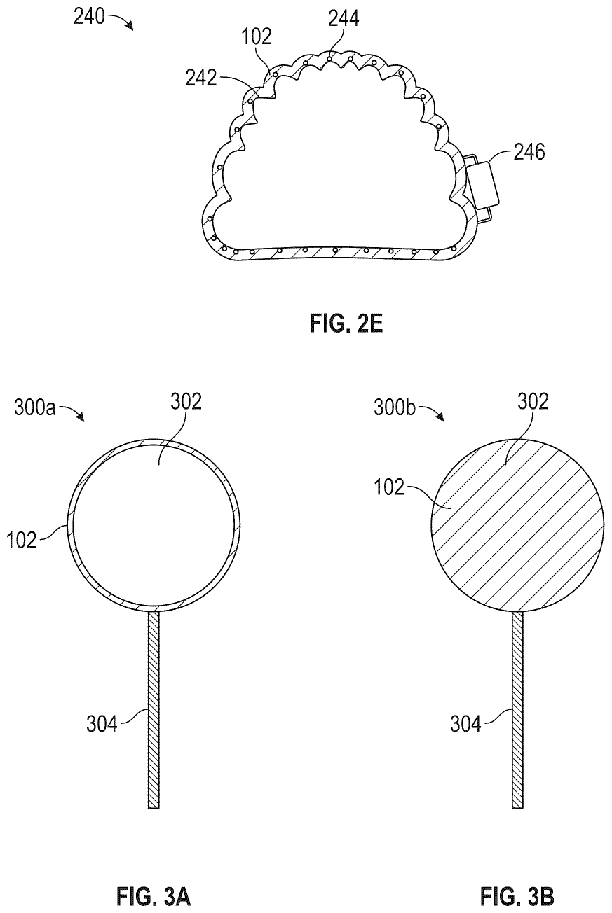

FIGS. 2A-2E are partially schematic illustrations of hydrogel configurations for dental appliances, in accordance with embodiments of the present technology. Any of the embodiments shown in FIGS. 2A-2E may be combined with each other and/or with the embodiment of FIGS. 1A and 1B.

FIG. 2A illustrates a pair of dental appliances 200a, 200b each including a hydrogel 102, in accordance with embodiments of the present technology. The dental appliances 200a, 200b can be configured to be worn on both jaws of the subject, e.g., the dental appliance 200a can include a shell 202 with cavities for receiving the teeth of the upper dental arch and the dental appliance 200b can include a shell 204 with cavities for receiving teeth of the lower dental arch. For instance, the dental appliances 200a, 200b can be a pair of aligners for the same treatment stage of a dental treatment plan and thus may be worn concurrently. The dental appliances 200a, 200b can have the same configuration of the hydrogel 102 (e.g., same type of hydrogel polymer, same type of detection agent(s), same hydrogel shape, same hydrogel location) or may have different configurations for the hydrogel 102 (e.g., different detection agents may be used if the upper jaw is expected to have different oral disease biomarkers than the lower jaw). In other embodiments, however, only one of the dental appliances 200a, 200b may include the hydrogel 102.

FIG. 2B illustrates a dental appliance 210 with distally-located hydrogels 102, in accordance with embodiments of the present technology. In the illustrated embodiment, the dental appliance 210 includes a shell 212 for receiving some or all of the teeth of a dental arch, the hydrogel 102 is located at the posterior buccal surfaces of the shell 212 (e.g., adjacent to one or more posterior teeth at both sides of the arch), and the remaining surfaces of the shell 212 do not include any hydrogel 102. This configuration may be used, for example, to detect oral disease biomarkers present in the posterior buccal mucosa. In other embodiments, however, the hydrogel 102 can be located at a different portion of the shell 212, such as the posterior lingual surfaces, anterior buccal surfaces, anterior lingual surfaces, etc.

FIG. 2C illustrates a dental appliance 220 including a palatal portion 222 with a hydrogel 102, in accordance with embodiments of the present technology. The dental appliance 220 can include a tooth receiving portion 224 (e.g., a shell) that receives some or all of the teeth of a subject's upper dental arch, and a palatal portion 222 extending between the left and right sides of the tooth receiving portion 224. For example, the dental appliance 220 can be a palatal expander, such that the palatal portion 222 applies forces to the left and right sides of the tooth receiving portion 224 to expand the subject's palate. Alternatively, the palatal portion 222 may not apply any treatment forces, and can be designed solely for carrying the hydrogel 102. When the appliance 220 is worn on the teeth, the palatal portion 222 with the hydrogel 102 can be placed proximate to or in contact with the subject's palate, thus allowing for detection of oral disease biomarkers located on or close proximity to the palate. The hydrogel 102 can be located on the surface of the palatal portion 222 facing the palate, the surface of the palatal portion 222 facing away from the palate, or both.

FIG. 2D illustrates a dental appliance 230 including a palatal portion 222 with a hydrogel 102 and projections 232, in accordance with embodiments of the present technology. The dental appliance 230 can be identical or generally similar to the dental appliance 220 of FIG. 2C, except that the palatal portion 222 of the dental appliance 230 includes a plurality of projections 232 (e.g., bumps, posts, ridges, blocks). The projections 232 can be coated with the hydrogel 102 or the projections 232 can be composed of the hydrogel 102 (e.g., the projections 232 can be droplets of the hydrogel 102). The projections 232 can be formed in the surface of the palatal portion 222 that faces the palate, the surface of the palatal portion 222 facing away from the palate, or both. This configuration can be used to increase the surface area of the hydrogel 102 that is exposed to physiologic fluids in the mouth.

FIG. 2E illustrates a dental appliance 240 including a hydrogel 102 that is configured for electrical impedance tomography, in accordance with embodiments of the present technology. The dental appliance 240 includes a shell 242, a hydrogel 102 extending along the gingival border of the shell 242, and a plurality of electrodes 244 (e.g., copper electrodes) embedded in the hydrogel 102. The electrodes 244 can be electrically coupled to each other and to an electronics unit 246 carried by the shell 242. The electronics unit 246 can include a power source (e.g., a battery), data storage, and electrical circuitry for applying a current to one or more electrodes 244 and measuring the resulting voltages at one or more electrodes 244. The voltage measurement may be analyzed by one or more software algorithms implemented by processing circuitry onboard the electronics unit 246 and/or an external device that is in communication with the electronics unit 246 (e.g., a mobile device, a smart appliance case, or other computing device). The measurements can be used for positional and/or force mapping of the dental appliance 240, e.g., to determine changes in 3D shape of the dental appliance 240 and/or changes in the forces applied to the teeth by the appliance 240. For instance, the shape and/or forces of the dental appliance 240 at rest versus the dental appliance 240 when applied to the teeth can be compared. In such embodiments, the hydrogel 102 can have properties that allow for adhesion or inclusion into the internal and/or external surfaces of the dental appliance 240, as well as sufficient integrity and/or self-healing properties to resist degradation from occlusal forces. The hydrogel 102 may include detection agents for oral disease biomarkers (e.g., to allow for both monitoring of appliance shape/force and detection of oral diseases or conditions), or may not include any detection agents (e.g., if only monitoring of appliance shape/force is desired).

In some embodiments, the hydrogels described herein are used with other types of oral devices besides dental appliances. For example, the oral device may be a lollipop, popsicle, candy, or other item that is designed to be placed in the mouth for sucking. Advantages of this approach may include a more user-friendly format (e.g., colorings and/or flavorings may be incorporated to improve user experience), easier manufacturing, and/or lower material cost (e.g., since the oral device does not need to perform other functions, such as applying forces to teeth, and thus can be a single use disposable).

FIG. 3A illustrates a lollipop-type oral device 300a (“lollipop device 300a”) including a hydrogel 102, in accordance with embodiments of the present technology. The lollipop device 300a includes an intraoral portion 302 configured to be inserted into a subject's mouth, and a support shaft 304 coupled to the intraoral portion 302 for the subject to hold. The hydrogel 102 can be coated onto the surface of the intraoral portion 302, e.g., via dip coating, spraying coating, spin coating, etc. Accordingly, when the subject sucks on the intraoral portion 302, the hydrogel 102 is exposed to physiologic fluids within the subject's mouth. The interior volume of the intraoral portion 302 can be made of any biocompatible material (e.g., a biocompatible polymer) suitable for acting as a substrate for coating the hydrogel 102 thereon. Although the intraoral portion 302 is depicted as having a circular cross-sectional shape, in other embodiments, the intraoral portion 302 can have a different cross-sectional shape, such as square, rectangular, oval, etc.

FIG. 3B illustrates a lollipop-type oral device 300b (“lollipop device 300b”) including a hydrogel 102, in accordance with embodiments of the present technology. The lollipop device 300b can be identical or generally similar to the lollipop device 300a of FIG. 3A, except that the intraoral portion 302 of the lollipop device 300b is composed of the hydrogel 102, such that the hydrogel 102 makes up both the surface and the interior volume of the intraoral portion 302.

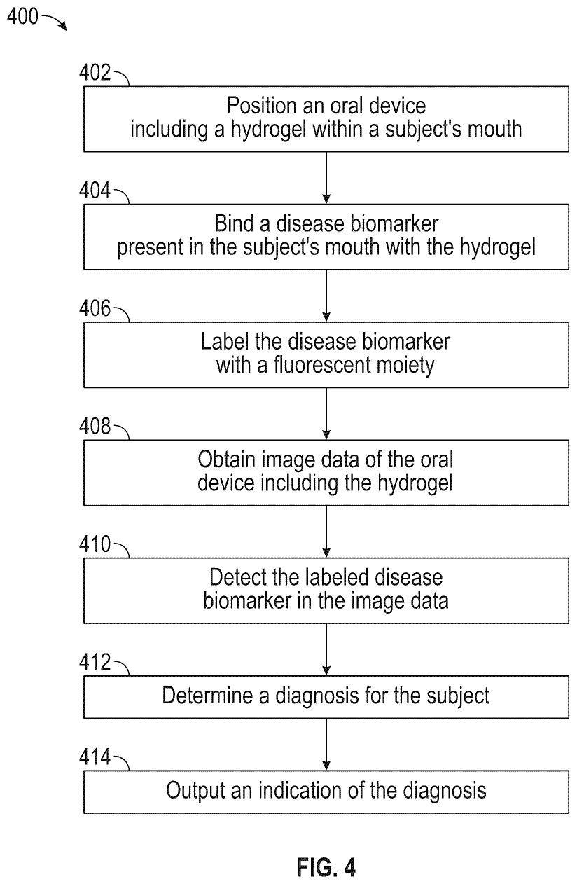

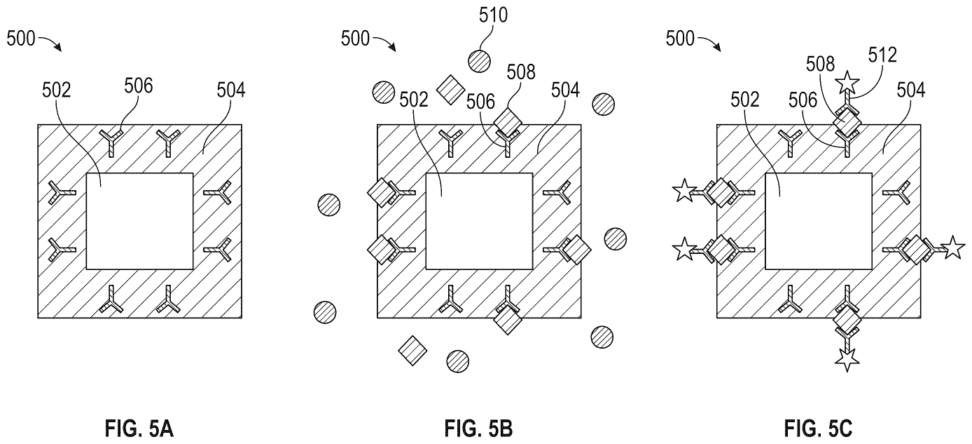

FIG. 4 is a flow diagram illustrating a method 400 for detecting an oral disease biomarker in a subject, in accordance with embodiments of the present technology. The method 400 can be used to diagnose a subject with any of the diseases or conditions described herein, and may be used in combination with any of the materials, devices, and systems described herein. Certain processes of the method 400 are described below in connection with FIGS. 5A-5C, which are schematic illustrations of a system 500 for detecting an oral disease biomarker in a subject, in accordance with embodiments of the present technology.

The method 400 can begin at block 402 with positioning an oral device including a hydrogel in a subject's mouth. The oral device and hydrogel can be part of a system (e.g., a kit) for detecting a disease or condition in the subject. For instance, as shown in FIG. 5A, the system 500 includes an oral device 502 and a hydrogel 504 carried by the oral device 502. The oral device 502 can have many different form factors, such as a dental appliance (e.g., as discussed with respect to FIGS. 1A-2E above and Section II below), a lollipop-type device (e.g., as discussed with respect to FIGS. 3A and 3B), or any other device suitable for being inserted partially or fully in the subject's mouth.

The hydrogel 504 can include any of the embodiments described herein, e.g., in Section I.A above. For example, the hydrogel can include one or more hydrophilic polymers that are covalently or non-covalently crosslinked to form a polymer network. The hydrogel 504 can be coupled to the oral device 502 (e.g., via bonding, coating, adhesives, fasteners) or can be integrally formed with the oral device 502 (e.g., the hydrogel 504 makes up a portion of or the entirety of the oral device 502). The hydrogel 504 can be located at any suitable portion of the oral device 502, such as an external surface of the oral device 502, an internal surface of the oral device 502, an interior volume of the oral device 502, etc.

In the illustrated embodiment, the hydrogel 504 includes a capture moiety 506 that is configured to bind (e.g., reversibly or irreversibly) to an oral disease biomarker 508 (FIGS. 5B and 5C). The capture moiety 506 and the oral disease biomarker 508 can be any of the embodiments described herein, e.g., in Sections I.B and I.C above, respectively. For example, the capture moiety 506 can be an antibody (e.g., a full-length antibody, an antibody fragment such as an Fab or scFv fragment), an aptamer, a peptide, a polysaccharide, or a small molecule. The capture moiety 506 can be encapsulated within and immobilized in the hydrogel 504, e.g., via covalent bonding to the polymer network of the hydrogel 504 and/or physical entrapment within the hydrogel 504.

Returning to FIG. 4, at block 404, the method 400 can include binding a disease biomarker that is present in the subject's mouth with the hydrogel. In some embodiments, the hydrogel includes a detection agent (e.g., a capture moiety) coupled to the polymer of the hydrogel, and the detection agent is configured to bind the disease biomarker.

For example, referring to FIG. 5B, when the oral device 502 is placed in the subject's mouth, the hydrogel 504 is exposed to physiological fluids in the mouth (e.g., saliva, GCF). The physiological fluids can include oral disease biomarkers 508 that are associated with a disease or condition of interest, as well as other species 510 (e.g., salivary proteins) that are not relevant to the disease or condition of interest. When the physiological fluids infiltrate into the hydrogel 504, the capture moieties 506 within the hydrogel 504 can bind specifically to the oral disease biomarkers 508 while exhibiting substantially no binding to the other species 510. In some embodiments, the oral device 502 remains in the subject's mouth for a sufficient amount of time for the capture moieties 506 to bind to the oral disease biomarkers 508, such as at least 1 minute, 2 minutes, 5 minutes, 10 minutes, 15 minutes, 20 minutes, 30 minutes, 45 minutes, 1 hour, 2 hours, 4 hours, 12 hours, or 24 hours.

Returning to FIG. 4, at block 406, the method 400 can continue with labeling the disease biomarker with a fluorescent moiety. In some embodiments, the fluorescent moiety is provided separately from the hydrogel and is applied to the hydrogel after the oral device has been removed from the subject's mouth.

For example, referring to FIG. 5C, the oral device 502 can be removed from the subject's mouth, along with the hydrogel 504 and the oral disease biomarkers 508 bound by the capture moieties 506. A fluorescent moiety 512 can then be applied to the hydrogel 504 to label the oral disease biomarkers 508 within the hydrogel 504. The fluorescent moiety 512 can be any of the embodiments described herein, e.g., in Section I.B above. For example, the fluorescent moiety 512 can include a fluorescent label (e.g., an organic dye, a polymer dye, a fluorescent protein, a quantum dot) that is coupled to a component (e.g., an antibody, an aptamer, a peptide, a polysaccharide, a small molecule) that binds to the oral disease biomarker 508.

The fluorescent moiety 512 may be applied to the hydrogel 504 in any suitable manner, such as immersing the hydrogel 504 in a solution of the fluorescent moiety 512; spraying, dipping, dripping, or brushing a solution of the fluorescent moiety 512 onto the hydrogel 504; etc. The hydrogel 504 may remain on the oral device 502 during the application of the fluorescent moiety 512, or the hydrogel 504 may be separated from the oral device 502 before the fluorescent moiety 512 is applied. The hydrogel 504 can be exposed to the fluorescent moiety 512 for a sufficient amount of time for the fluorescent moieties 512 to bind to the oral disease biomarkers 508, such as at least 1 minute, 2 minutes, 5 minutes, 10 minutes, 15 minutes, 20 minutes, 30 minutes, 45 minutes, 1 hour, 2 hours, 4 hours, 12 hours, or 24 hours. Any unbound fluorescent moiety 512 can be washed off the hydrogel 504.

Returning to FIG. 4, at block 408, the method 400 can include obtaining image data of the oral device, including the hydrogel with the labeled disease biomarker. The image data can include photographs, videos, scans, or combinations thereof. The image data can include fluorescent images only or may include images obtained using other modalities (e.g., color images, scan data). The image data can be generated using any imaging device having fluorescent imaging capabilities. For example, a scanner (e.g., an intraoral scanner or an extraoral scanner) may be used to obtain image data of the oral device and hydrogel. The scanner can be configured to obtain images using multiple imaging modalities, such as fluorescent images, color images, and/or scans. Switching between the different imaging modalities can be implemented using hardware and/or software components. Representative examples of intraoral scanners that may be used are described in Section III below. As another example, a camera may be used to obtain image data of the oral device and hydrogel, such as a DLSR camera or mobile device camera configured with the appropriate hardware and/or software for fluorescent imaging (e.g., light sources and/or filters for producing the appropriate excitation wavelengths and imaging the appropriate emission wavelengths, software for switching between color imaging and fluorescent imaging modes).

At block 410, the method 400 can continue with detecting the labeled disease biomarker in the image data. In some embodiments, the detection involves analyzing the image data to determine whether a fluorescent signal corresponding to the fluorescent moiety is present in the image data, thereby indicating that the labeled disease biomarker is present in the hydrogel of the oral device (and thus was present in the subject's mouth). The detection can also include measuring a magnitude of the fluorescent signal, which may correspond to an amount of the labeled disease biomarker that is present. Optionally, the detection can include identifying a location of the fluorescent signal in the image data, and then using the identified location to determine the location of the labeled disease biomarker relative to the oral device, which may indicate where the disease biomarker was present in the mouth and thus the location of a disease or condition. The detection can be performed automatically using a software algorithm (e.g., using computer vision and/or machine learning techniques), manually based on user input (e.g., a user may view the image data and indicate whether the labeled disease biomarker is present), or a combination thereof (e.g., a software algorithm may generate an initial detection for review and/or approval by a user).

In embodiments where the image data includes scans of the oral device obtained using an intraoral or extraoral scanner, the scans may be used to produce a digital representation of the geometry of the oral device (e.g., using a registration algorithm to stitch together the scans to produce a 3D model and/or a 2D texture map of the oral device). The fluorescent images can then be overlaid onto the digital representation of the oral device, thereby showing how the fluorescent signal is spatially distributed with respect to the geometry of the oral device. Optionally, other types of images such as color images can also be overlaid onto the digital representation of the oral device, e.g., as part of the same overlay as the fluorescent images or as a different overlay that may be toggled on or off.

At block 412, the method 400 can include determining a diagnosis for the subject. The diagnosis can be based on the presence of the labeled disease biomarker in the image data, e.g., the subject is diagnosed as having a particular disease or condition or being at risk of developing the disease or condition if the associated disease biomarker is present in the image data. Optionally, the diagnosis can also be based on the magnitude of the fluorescent signal in the image data (e.g., the maximum fluorescent signal value, the average fluorescent signal value) which may correlate to the amount of the disease biomarker that is present in the subject's mouth (e.g., a positive diagnosis may be made only if the fluorescent signal/biomarker amount is above a predetermined threshold). The diagnosis may also be based on the location of the fluorescent signal in the image data, which may correlate to the location of the disease biomarker in the subject's mouth (e.g., a positive diagnosis may be made only if the fluorescent signal/biomarker is present at a particular location, a negative diagnosis can be made if the fluorescent signal/biomarker is detected at a location that is unlikely or impossible to the site of a disease or condition (which may indicate a false positive signal)).

Optionally, the diagnosis may be based on additional data besides the image data. The additional data can be provided in any suitable format, such as 3D models, 2D images, graphs, tables, charts, numerical values, qualitative descriptors, etc. The additional data can be received from various data sources, such as electronic health records (e.g., dental records), databases, imaging systems, etc. The additional data can be any data type that provides information relevant to diagnosing the subject with a particular disease or condition.

For example, in some embodiments, the diagnosis may be based on previous image data of a labeled biomarker in an oral device, where the oral device has the same or similar configuration as the oral device used to obtain the current image data (e.g., includes the same hydrogel, capture moiety, and fluorescent moiety). The previous image data may be obtained at a previous time point that is at least 1 day, 2 days, 3 days, 4 days, 5 days, 6 days, 1 week, 2 weeks, 3 weeks, 4 weeks, 1 month, 2 months, 3 months, 6 months, 1 year, or more before the time point of the current image data. Optionally, the previous image data can include multiple sets of images that were obtained at multiple previous time points. The previous image data may be compared to the current image data to identify changes in the presence, amount, and/or location of the disease biomarker over time. For instance, a positive diagnosis may be made if the disease biomarker is present at the same location in the oral device over time and/or if the amount of the disease biomarker increases consistently over time. Conversely, a negative diagnosis may be made if the disease biomarker is not consistently present over time and/or if the amount of the disease biomarker varies significantly or decreases over time.

Alternatively or in combination, the diagnosis may also be based on other types of data, such as radiographic data (e.g., standard x-ray data such as bitewing x-ray data, panoramic x-ray data, cephalometric x-ray data, computed tomography (CT) data, cone-beam computed tomography (CBCT) data, fluoroscopy data), intraoral scan data, photographs, videos, demographic data (e.g., age, gender, race/ethnicity), medical history data (e.g., previous diagnoses, sexually transmitted diseases, smoking/vaping, quid/betel nut chewing, familial history of disease), etc. For example, the threshold for a positive diagnosis based on fluorescent images may be lowered if the subject is determined to be at high risk for developing a particular disease or condition based on their demographic data and/or medical history data. As another example, the fluorescent images may be compared to images obtained using other imaging modalities (e.g., radiographic images, color images, NIR images) to determine if the disease biomarker locations identified in the fluorescent images are consistent with the potential disease locations identified in the other image. A positive diagnosis may be made if the disease locations determined from multiple imaging modalities are consistent with each other, while a negative or inconclusive diagnosis may be made if the disease locations determined from multiple imaging modalities are inconsistent. Optionally, a positive diagnosis may be made as long as multiple imaging modalities indicate the presence of the same disease or condition, without requiring that the disease location be the same across the different imaging modalities.

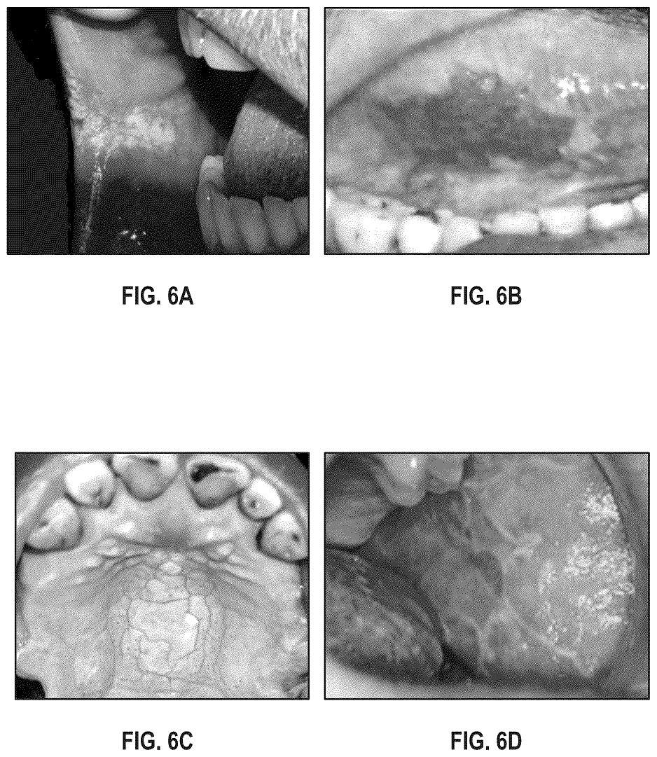

For example, FIGS. 6A-6D are photographs illustrating representative examples of precancerous lesions in the mouth, in accordance with embodiments of the present technology. FIG. 6A depicts leukoplakia, which is the most common precancerous lesion in the mouth and presents as whitish patches/lesions of oral mucosa that cannot be removed by scraping. High risk areas for leukoplakia include the underside of tongue, the floor of the mouth, and the soft palate. FIG. 6B depicts erythroplakia, which is a less common precancerous lesion (but more prone to show the presence of dysplasia) and presents as a red raised lesion on mucous membranes. High risk areas for erythroplakia include the underside of the tongue, the floor of the mouth, and the buccal mucosa. FIG. 6C depicts palatal lesions caused by placing the lit end of a cigarette or cigar inside the mouth. FIG. 6D depicts oral lichen planus, which is a pre-malignant condition that presents as white lacy patterns or threads on the buccal mucosa or tongue. Oral lichen planus may be classified as reticular (white patches) or erosive (bright red gums). Other types of oral lesions that may be associated with oral cancer include hyperkeratosis, a white thickening of the mucosa that is not typically pre-malignant but is often seen in subjects who use smokeless tobacco; dyskeratosis congenital (DC), and epidermolysis bullosa. In some embodiments, the subject is diagnosed as having oral cancer or being at risk of developing oral cancer if precancerous lesions are detected in color images of the subject's mouth and if the fluorescent images indicate the presence of oral cancer biomarkers (and optionally, if the oral cancer biomarkers are present at locations in the oral device corresponding to the locations of the precancerous lesions in the mouth).

At block 414, the method 400 can include outputting an indication of the diagnosis. The indication can be output to a user (e.g., the subject, a clinician) via a user interface of a display of a computing device (e.g., a monitor or touch screen of a mobile device (such as a smartphone), tablet, laptop, workstation, etc.). The indication of the diagnosis can be presented in many different formats, such as numerically (e.g., scores, probabilities), textually (e.g., categorizations, descriptive assessments, notifications), and/or graphically. For instance, the indication can state the type of disease or condition that the subject is diagnosed with and a confidence level associated with the diagnosis. Alternatively, the indication may simply state that a particular disease biomarker was detected (and, optionally, the amount and/or location of the disease biomarker).

In some embodiments, the process of block 414 includes outputting one or more fluorescent images showing the labeled disease biomarker. The fluorescent images may be presented as individual images or as an overlay on a digital representation (e.g., 3D model) of the oral device (e.g., generated from scan data of the oral device as discussed above). Optionally, the overlay can also include other types of image data, such as color images, NIR images, etc. The user interface can allow the user to toggle between viewing the 3D model and individual 2D images, and/or between viewing images/overlays obtained with different imaging modalities. This approach can be advantageous, for example, to assist a user in visually identifying the spatial location of the detected disease biomarkers and/or the spatial relationships between the disease biomarkers and disease sites that are visible in other imaging modalities.

Optionally, if the subject is diagnosed with a disease or condition and/or if the subject is determined to have an abnormal level of a disease biomarker (e.g., a level falling outside of a normal range and/or within a range associated with a disease or condition), the method 400 can further include outputting a treatment recommendation. The treatment recommendation can include one or more actions to be performed by the clinician and/or patient to address the positive diagnosis and/or abnormal biomarker level. For instance, a positive diagnosis and/or an abnormal biomarker level may produce a recommendation that the clinician perform additional diagnostic procedures on that location (e.g., visual inspection, biopsy) to confirm that that the diagnosis is accurate.

The method 400 illustrated in FIG. 4 can be modified in many different ways. For example, the ordering of the processes shown in FIG. 4 can be varied. Some of the processes of the method 400 can be omitted (e.g., the processes of blocks 412 and/or 414), and/or the method 400 can include processes not shown in FIG. 4. Some of the processes of the method 400 may be performed by a user (e.g., a clinician or the subject), such as the processes of blocks 402-408. Some of the processes of the method 400 may be implemented as computer-readable instructions (e.g., program code) that are configured to be executed by one or more processors of a computing device (e.g., a client device, a server device, or suitable combinations thereof), such as the processes of blocks 410-414.

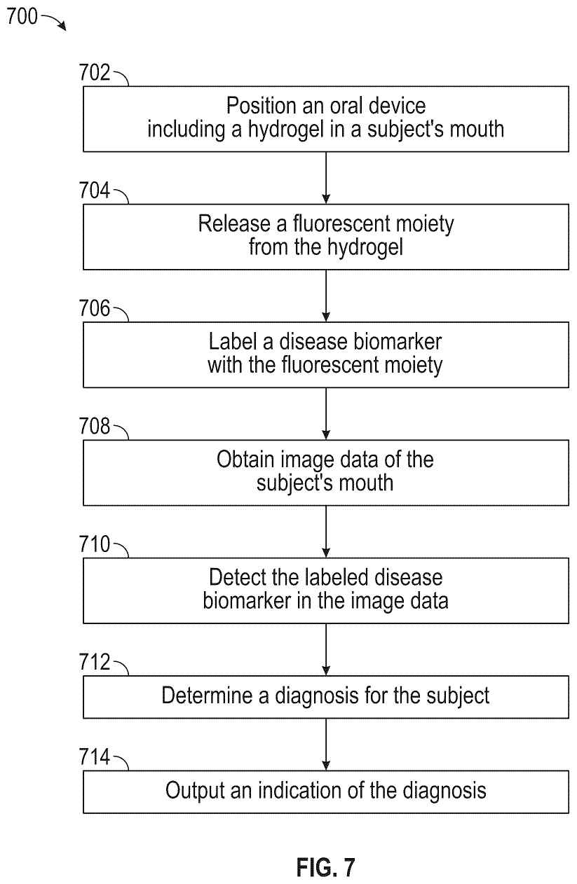

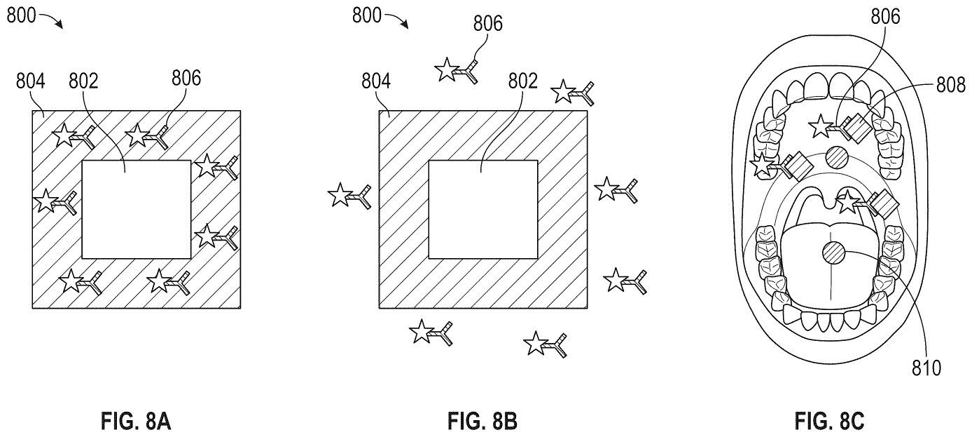

FIG. 7 is a flow diagram illustrating a method 700 for detecting an oral disease biomarker in a subject, in accordance with embodiments of the present technology. The method 700 can be used to diagnose a subject with any of the diseases or conditions described herein, and may be used in combination with any of the materials, devices, and systems described herein. Certain processes of the method 700 are described below in connection with FIGS. 8A-8C, which are schematic illustrations of a system 800 for detecting a disease or condition in a subject, in accordance with embodiments of the present technology.

The method 700 can begin at block 702 with positioning an oral device including a hydrogel in a subject's mouth. The oral device and hydrogel can be part of a system (e.g., a kit) for detecting a disease or condition in the subject. For instance, as shown in FIG. 8A, the system 800 includes an oral device 802 and a hydrogel 804 carried by the oral device 802. The oral device 802 can have many different form factors, such as a dental appliance (e.g., as discussed with respect to FIGS. 1A-2E above and Section II below), a lollipop-type device (e.g., as discussed with respect to FIGS. 3A and 3B), or any other device suitable for being inserted partially or fully in the subject's mouth.

The hydrogel 804 can include any of the embodiments described herein, e.g., in Section I.A above. For example, the hydrogel can include one or more hydrophilic polymers that are covalently or non-covalently crosslinked to form a polymer network. The hydrogel 804 can be coupled to the oral device 802 (e.g., via bonding, coating, adhesives, fasteners) or can be integrally formed with the oral device 802 (e.g., the hydrogel 804 makes up a portion of or the entirety of the oral device 802). The hydrogel 804 can be located at any suitable portion of the oral device 802, such as an external surface of the oral device 802, an internal surface of the oral device 802, an interior volume of the oral device 802, etc.

In the illustrated embodiment, the hydrogel 804 includes a fluorescent moiety 806 that is configured to bind (e.g., reversibly or irreversibly) to and label an oral disease biomarker 808 (FIG. 8C). The fluorescent moiety 806 and the oral disease biomarker 808 can be any of the embodiments described herein, e.g., in Sections I.B and I.C above, respectively. For example, the fluorescent moiety 806 can include a fluorescent label (e.g., an organic dye, a polymer dye, a fluorescent protein, a quantum dot) that is coupled to a component (e.g., an antibody, an aptamer, a peptide, a polysaccharide, a small molecule) that binds to the oral disease biomarker 808. The fluorescent moiety 806 can be encapsulated within the hydrogel 804 in a manner that allows the fluorescent moiety 806 to be released from the hydrogel 804 (e.g., the fluorescent moiety 806 is not conjugated to the polymer of the hydrogel 804).

Returning to FIG. 7, at block 704, the method 700 can include releasing a fluorescent moiety from the hydrogel. The release of the fluorescent moiety can be due to diffusion of the fluorescent moiety into physiological fluids in the mouth and/or can be triggered when the hydrogel is exposed to a stimulus (e.g., physiological temperature, pH, ionic strength).

At block 706, the method 700 can include labeling a disease biomarker with the fluorescent moiety. For example, as shown in FIG. 8B, when the oral device 802 is placed in the subject's mouth, the hydrogel 804 is exposed to physiological fluids in the mouth (e.g., saliva, GCF). The physiological fluids can include oral disease biomarkers 808 that are associated with a disease or condition of interest, as well as other species 810 (e.g., salivary proteins) that are not relevant to the disease or condition of interest. The fluorescent moieties 806 within the hydrogel 804 can be released into the physiological fluids and can bind specifically to the oral disease biomarkers 808 while exhibiting substantially no binding to the other species 810. In some embodiments, the oral device 802 remains in the subject's mouth for a sufficient amount of time for the fluorescent moieties 806 to be released and to bind to the oral disease biomarkers 808, such as at least 1 minute, 2 minutes, 5 minutes, 10 minutes, 15 minutes, 20 minutes, 30 minutes, 45 minutes, 1 hour, 2 hours, 4 hours, 12 hours, or 24 hours.

Referring next to FIG. 8C, after the desired time period has elapsed, the oral device 802 can be removed from the subject's mouth along with the hydrogel 804, thus leaving the fluorescent moieties 806 bound to the oral disease biomarkers 808 in the subject's mouth. Optionally, the subject's mouth can be rinsed to remove any unbound fluorescent moieties 806.

Returning to FIG. 7, at block 708, the method 700 can include obtaining image data of the subject's mouth, including the labeled disease biomarker. The image data can include photographs, videos, scans, or combinations thereof. The image data can include fluorescent images only or may include images obtained using other modalities (e.g., color images, scan data). The image data can be generated using any imaging device having fluorescent imaging capabilities. For example, a scanner (e.g., an intraoral scanner) may be used to obtain image data of the subject's mouth (e.g., buccal mucosa, lips, teeth, gingiva, tongue, hard palate, soft palate, retromolar trigone, tonsil, uvula, floor of the mouth, throat). The scanner can be configured to obtain images using multiple imaging modalities, such as fluorescent images, color images, and/or scan data. Switching between the different imaging modalities can be implemented using hardware and/or software components. Representative examples of intraoral scanners that may be used are described in Section III below. As another example, a camera may be used to obtain image data of the subject's mouth, such as a DLSR camera or mobile device camera configured with the appropriate hardware and/or software for fluorescent imaging (e.g., light sources and/or filters for producing the appropriate excitation wavelengths and imaging the appropriate emission wavelengths, software for switching between color imaging and fluorescent imaging modes). Optionally, accessories such as cheek retractors may be used to facilitate imaging of the mouth.

At block 710, the method 700 can continue with detecting the labeled disease biomarker in the image data. In some embodiments, the detection involves analyzing the image data to determine whether a fluorescent signal corresponding to the fluorescent moiety is present in the image data, thereby indicating that the labeled disease biomarker is present in the subject's mouth. The detection can also include measuring a magnitude of the fluorescent signal, which may correspond to an amount of the labeled disease biomarker that is present. Optionally, the detection can include identifying a location of the fluorescent signal in the image data, and then using the identified location to determine the location of the labeled disease biomarker in the mouth and thus the location of a disease or condition. The detection can be performed automatically using a software algorithm (e.g., using computer vision and/or machine learning techniques), manually based on user input (e.g., a user may view the image data and indicate whether the labeled disease biomarker is present), or a combination thereof (e.g., a software algorithm may generate an initial detection for review and/or approval by a user).

In embodiments where the image data includes scans of the subject's mouth obtained using an intraoral scanner, the scans may be used to produce a digital representation of the tissues of the mouth (e.g., using a registration algorithm to stitch together the scans to produce a 3D model and/or a 2D texture map of the mouth). The fluorescent images can then be overlaid onto the digital representation of tissues of the mouth, thereby showing how the fluorescent signal is spatially distributed with respect to the tissues of the mouth. Optionally, other types of images such as color images can also be overlaid onto the digital representation of the tissues of the mouth e.g., as part of the same overlay as the fluorescent images or as a different overlay that may be toggled on or off.

At block 712, the method 700 can include determining a diagnosis for the subject. The process of block 712 may be identical or generally similar to the process of block 412 of the method 400 of FIG. 4. For example, the diagnosis can be based on the presence, amount, and/or location of the labeled disease biomarker in the image data. The diagnosis can also be based on other types of data, such as radiographic data (e.g., x-ray data), intraoral scan data, photographs, videos, demographic data (e.g., age, gender, race/ethnicity), medical history data (e.g., previous diagnoses, sexually transmitted diseases, smoking/vaping, quid/betel nut chewing, familial history of disease), etc.

Optionally, the diagnosis may be based on previous image data of a labeled biomarker in the subject's mouth, where the labeled biomarker was released from an oral device having the same or similar configuration as the oral device used to obtain the current image data (e.g., includes the same hydrogel and fluorescent moiety). The previous image data may be obtained at a previous time point that is at least 1 day, 2 days, 3 days, 4 days, 5 days, 6 days, 1 week, 2 weeks, 3 weeks, 4 weeks, 1 month, 2 months, 3 months, 6 months, 1 year, or more before the time point of the current image data. Optionally, the previous image data can include multiple sets of images that were obtained at multiple previous time points. The previous image data may be compared to the current image data to identify changes in the presence, amount, and/or location of the disease biomarker over time. For instance, a positive diagnosis may be made if the disease biomarker is present at the same location in the mouth over time and/or if the amount of the disease biomarker increases consistently over time. Conversely, a negative diagnosis may be made if the disease biomarker is not consistently present over time and/or if the amount of the disease biomarker varies significantly or decreases over time.

At block 714, the method 700 can include outputting an indication of a diagnosis for the subject. The process of block 714 may be identical or generally similar to the process of block 414 of the method 400 of FIG. 4. For instance, the indication can include the type, amount, and/or location of the detected disease biomarker; the type of disease or condition that the subject is diagnosed with; a confidence level associated with the diagnosis; and/or treatment recommendations for the diagnosis.

In some embodiments, the process of block 714 includes outputting one or more fluorescent images showing the labeled disease biomarker. The fluorescent images may be presented as individual images or as an overlay on a digital representation (e.g., 3D model) of the subject's mouth (e.g., generated from scan data of the mouth as discussed above). Optionally, the overlay can also include other types of image data, such as color images, NIR images, etc. The user interface can allow the user to toggle between viewing the 3D model and individual 2D images, and/or between viewing images/overlays obtained with different imaging modalities. This approach can be advantageous, for example, to assist a user in visually identifying the spatial location of the detected disease biomarkers and/or the spatial relationships between the disease biomarkers and disease sites that are visible in other imaging modalities.

The method 700 illustrated in FIG. 7 can be modified in many different ways. For example, the ordering of the processes shown in FIG. 7 can be varied. Some of the processes of the method 700 can be omitted (e.g., the processes of blocks 712 and/or 714), and/or the method 700 can include processes not shown in FIG. 7. Some of the processes of the method 700 may be performed by a user (e.g., a clinician or the subject), such as the processes of blocks 702-708. Some of the processes of the method 700 may be implemented as computer-readable instructions (e.g., program code) that are configured to be executed by one or more processors of a computing device (e.g., a client device, a server device, or suitable combinations thereof), such as the processes of blocks 710-714.

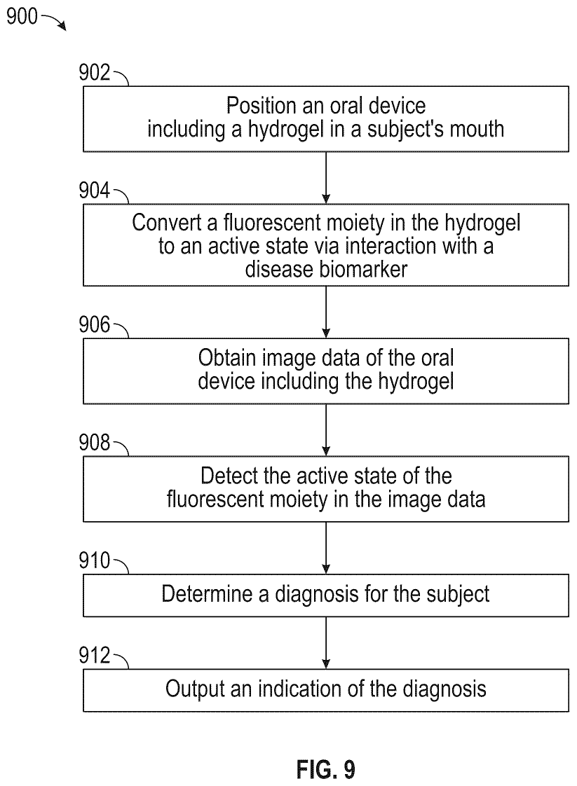

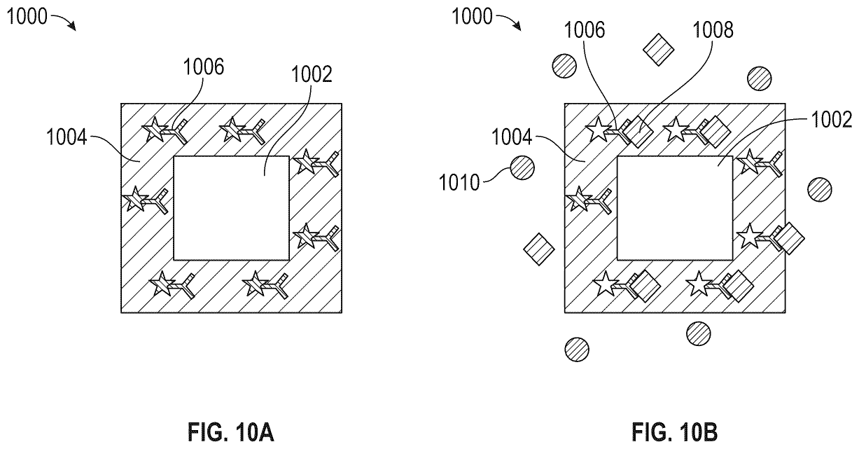

FIG. 9 is a flow diagram illustrating a method 900 for detecting an oral disease biomarker in a subject, in accordance with embodiments of the present technology. The method 900 can be used to diagnose a subject with any of the diseases or conditions described herein, and may be used in combination with any of the materials, devices, and systems described herein. Certain processes of the method 900 are described below in connection with FIGS. 10A and 10B, which are schematic illustrations of a system 1000 for detecting a disease or condition in a subject, in accordance with embodiments of the present technology.

The method 900 can begin at block 902 with positioning an oral device including a hydrogel in a subject's mouth. The oral device and hydrogel can be part of a system (e.g., a kit) for detecting a disease or condition in the subject. For instance, as shown in FIG. 10A, the system 1000 includes an oral device 1002 and a hydrogel 1004 carried by the oral device 1002. The oral device 1002 can have many different form factors, such as a dental appliance (e.g., as discussed with respect to FIGS. 1A-2E above and Section II below), a lollipop-type device (e.g., as discussed with respect to FIGS. 3A and 3B), or any other device suitable for being inserted partially or fully in the subject's mouth.

The hydrogel 1004 can include any of the embodiments described herein, e.g., in Section I.A above. For example, the hydrogel can include one or more hydrophilic polymers that are covalently or non-covalently crosslinked to form a polymer network. The hydrogel 1004 can be coupled to the oral device 1002 (e.g., via bonding, coating, adhesives, fasteners) or can be integrally formed with the oral device 1002 (e.g., the hydrogel 1004 makes up a portion of or the entirety of the oral device 1002). The hydrogel 1004 can be located at any suitable portion of the oral device 1002, such as an external surface of the oral device 1002, an internal surface of the oral device 1002, an interior volume of the oral device 1002, etc.

In the illustrated embodiment, the hydrogel 1004 includes a fluorescent moiety 1006 that is configured to bind (e.g., reversibly or irreversibly) to and label an oral disease biomarker 1008 (FIG. 10B). The fluorescent moiety 1006 and the oral disease biomarker 1008 can be any of the embodiments described herein, e.g., in Sections I.B and I.C above, respectively. For example, the fluorescent moiety 1006 can include a fluorescent label (e.g., an organic dye, a polymer dye, a fluorescent protein, a quantum dot) that is coupled to a component (e.g., an antibody, an aptamer, a peptide, a polysaccharide, a small molecule) that binds to the oral disease biomarker 1008. In some embodiments, the fluorescent moiety 1006 is encapsulated within and immobilized in the hydrogel 1004, e.g., via covalent bonding to the polymer network of the hydrogel 1004 and/or physical entrapment within the hydrogel 1004. In other embodiments, however, the fluorescent moiety 1006 is encapsulated within the hydrogel 1004 in a manner that allows the fluorescent moiety 1006 to be released from the hydrogel 1004 (e.g., the fluorescent moiety 1006 is not conjugated to the polymer of the hydrogel 1004).

Returning to FIG. 9, at block 904, the method 900 can include converting a fluorescent moiety in the hydrogel to an active state via interaction with a disease biomarker. As shown in FIG. 10A, the fluorescent moiety 1006 can be a smart molecule that is initially present in an inactive (e.g., non-fluorescent) state. Referring next to FIG. 10B, when the oral device 1002 is placed in the subject's mouth, the hydrogel 1004 is exposed to physiological fluids in the mouth (e.g., saliva, GCF). The physiological fluids can include oral disease biomarkers 1008 that are associated with a disease or condition of interest, as well as other species 1010 (e.g., salivary proteins) that are not relevant to the disease or condition of interest. When the physiological fluids infiltrate into the hydrogel 1004, the fluorescent moieties 1006 within the hydrogel 1004 can bind specifically to the oral disease biomarkers 1008 while exhibiting substantially no binding to the other species 1010. The binding of the oral disease biomarkers 1008 to the fluorescent moieties 1006 can convert the fluorescent moieties 1006 to an active (e.g., fluorescent) state, e.g., via an enzymatic reaction, a conformational change, removal of a fluorescence quencher, etc. In some embodiments, the oral device 1002 remains in the subject's mouth for a sufficient amount of time for the fluorescent moieties 1006 to bind to the oral disease biomarkers 1008 and to be converted into the active state, such as at least 1 minute, 2 minutes, 5 minutes, 10 minutes, 15 minutes, 20 minutes, 30 minutes, 45 minutes, 1 hour, 2 hours, 4 hours, 12 hours, or 24 hours.

Returning to FIG. 9, at block 906, the method 900 can include obtaining image data of the oral device, including the hydrogel with the fluorescent moiety. The image data can be obtained while the oral device remains in the subject's mouth or can be obtained after the oral device has been removed from the subject's mouth. The image data can include photographs, videos, scans, or combinations thereof. The image data can include fluorescent images only or may include images obtained using other modalities (e.g., color images, scan data). The image data can be generated using any imaging device having fluorescent imaging capabilities. For example, a scanner (e.g., an intraoral scanner or an extraoral scanner) may be used to obtain image data of the oral device and hydrogel. The scanner can be configured to obtain images using multiple imaging modalities, such as fluorescent images, color images, and/or scans. Switching between the different imaging modalities can be implemented using hardware and/or software components. Representative examples of intraoral scanners that may be used are described in Section III below. As another example, a camera may be used to obtain image data of the oral device and hydrogel, such as a DLSR camera or mobile device camera configured with the appropriate hardware and/or software for fluorescent imaging (e.g., light sources and/or filters for producing the appropriate excitation wavelengths and imaging the appropriate emission wavelengths, software for switching between color imaging and fluorescent imaging modes).

At block 908, the method 900 can continue with detecting the active state of the fluorescent moiety in the image data. In some embodiments, the detection involves analyzing the image data to determine whether a fluorescent signal corresponding to the active state of the fluorescent moiety is present in the image data, thereby indicating that the disease biomarker interacted with the hydrogel of the oral device (and thus was present in the subject's mouth). The detection can also include measuring a magnitude of the fluorescent signal, which may correspond to an amount of the disease biomarker that is present. Optionally, the detection can include identifying a location of the fluorescent signal in the image data, and then using the identified location to determine the location of the disease biomarker relative to the oral device, which may indicate where the disease biomarker was present in the mouth and thus the location of a disease or condition. The detection can be performed automatically using a software algorithm (e.g., using computer vision and/or machine learning techniques), manually based on user input (e.g., a user may view the image data and indicate whether the active state of the fluorescent moiety is present), or a combination thereof (e.g., a software algorithm may generate an initial detection for review and/or approval by a user).

In embodiments where the image data includes scans of the oral device obtained using an intraoral or extraoral scanner, the scans may be used to produce a digital representation of the geometry of the oral device (e.g., using a registration algorithm to stitch together the scans to produce a 3D model and/or a 2D texture map of the oral device). The fluorescent images can then be overlaid onto the digital representation of the oral device, thereby showing how the fluorescent signal is spatially distributed with respect to the geometry of the oral device. Optionally, other types of images such as color images can also be overlaid onto the digital representation of the oral device, e.g., as part of the same overlay as the fluorescent images or as a different overlay that may be toggled on or off.

At block 910, the method 900 can include determining a diagnosis for the subject. The process of block 910 may be identical or generally similar to the process of block 412 of the method 400 of FIG. 4. For example, the diagnosis can be based on the presence, amount, and/or location of the active fluorescent moiety in the image data. The diagnosis can also be based on other types of data, such as radiographic data (e.g., x-ray data), intraoral scan data, photographs, videos, demographic data (e.g., age, gender, race/ethnicity), medical history data (e.g., previous diagnoses, sexually transmitted diseases, smoking/vaping, quid/betel nut chewing, familial history of disease), etc.

Optionally, the diagnosis may be based on previous image data of an oral device in the subject's mouth, where the oral device has the same or similar configuration as the oral device used to obtain the current image data (e.g., includes the same hydrogel and fluorescent moiety). The previous image data may be obtained at a previous time point that is at least 1 day, 2 days, 3 days, 4 days, 5 days, 6 days, 1 week, 2 weeks, 3 weeks, 4 weeks, 1 month, 2 months, 3 months, 6 months, 1 year, or more before the time point of the current image data. Optionally, the previous image data can include multiple sets of images that were obtained at multiple previous time points. The previous image data may be compared to the current image data to identify changes in the presence, amount, and/or location of the active fluorescent moiety over time. For instance, a positive diagnosis may be made if the active fluorescent moiety is present at the same location in the oral device over time and/or if the amount of the active fluorescent moiety increases consistently over time. Conversely, a negative diagnosis may be made if the active fluorescent moiety is not consistently present over time and/or if the amount of the active fluorescent moiety varies significantly or decreases over time.

At block 912, the method 900 can include outputting an indication of a diagnosis for the subject. The process of block 912 may be identical or generally similar to the process of block 414 of the method 400 of FIG. 4. For instance, the indication can include the type, amount, and/or location of the detected active fluorescent moiety and/or disease biomarker; the type of disease or condition that the subject is diagnosed with; a confidence level associated with the diagnosis; and/or treatment recommendations for the diagnosis.

In some embodiments, the process of block 912 includes outputting one or more fluorescent images showing the active fluorescent moiety. The fluorescent images may be presented as individual images or as an overlay on a digital representation (e.g., 3D model) of the oral device (e.g., generated from scan data of the oral device as discussed above). Optionally, the overlay can also include other types of image data, such as color images, NIR images, etc. The user interface can allow the user to toggle between viewing the 3D model and individual 2D images, and/or between viewing images/overlays obtained with different imaging modalities. This approach can be advantageous, for example, to assist a user in visually identifying the spatial location of the active fluorescent moiety (and thus, the disease biomarker) and/or the spatial relationships between the disease biomarkers and disease sites that are visible in other imaging modalities.

The method 900 illustrated in FIG. 9 can be modified in many different ways. For example, the ordering of the processes shown in FIG. 9 can be varied. Some of the processes of the method 900 can be omitted (e.g., the processes of blocks 910 and/or 912), and/or the method 900 can include processes not shown in FIG. 9. Some of the processes of the method 900 may be performed by a user (e.g., a clinician or the subject), such as the processes of blocks 902-906. Some of the processes of the method 900 may be implemented as computer-readable instructions (e.g., program code) that are configured to be executed by one or more processors of a computing device (e.g., a client device, a server device, or suitable combinations thereof), such as the processes of blocks 908-912.

Although certain embodiments of the present technology are described with respect to hydrogels that are carried by oral devices, this is not intended to be limiting, and the hydrogels herein can be used in other formats, such as in devices that are not inserted into the subject's mouth. For instance, the hydrogels herein can be applied to a collection container (e.g., via coating of the internal surface of the container) and the subject can expectorate into the collection container for detection of oral disease biomarkers in the saliva. As another example, the hydrogels herein can be applied directly to the subject's mouth to capture oral disease biomarkers, the subject can expectorate the hydrogel into a collection container, and the expectorated hydrogel can be analyzed to detect the captured biomarkers. In a further example, a subject may expectorate saliva into a collection container, and the saliva may subsequently be applied to a hydrogel for detection of oral disease biomarkers in the saliva.

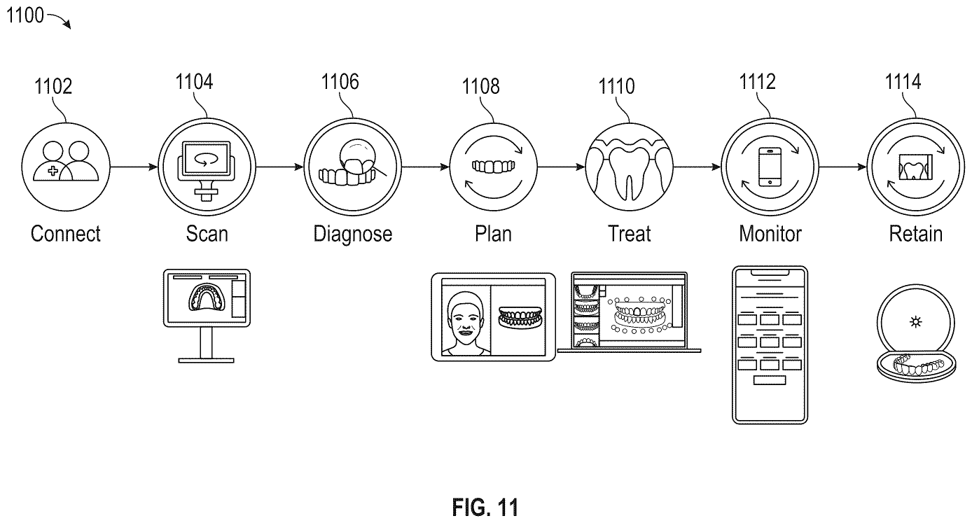

FIG. 11 illustrates a digital dental workflow 1100 for monitoring and/or treating a subject's teeth, in accordance with embodiments of the present technology. The workflow 1100 can incorporate any of the systems, devices, and methods described herein (e.g., with respect to FIGS. 1A-10B above). The workflow 1100 can include some or all of the following stages: connecting to a subject in need of dental treatment (connection stage 1102), scanning the subject's dentition to generate a digital representation (e.g., 3D model) of the dentition (scanning stage 1104), generating an initial diagnosis of the subject's dentition (diagnosis stage 1106), generating one or more dental treatment plans for the subject's dentition (e.g., orthodontic, restorative, and/or diagnostic treatments) (planning stage 1108), implementing the treatment dental treatment plan (treatment stage 1110), monitoring treatment progress (monitoring stage 1112), and/or retaining the subject's dentition in a desired arrangement and/or other monitoring post-treatment (retention stage 1114).

Any of the stages of the workflow 1100 can include delivering a hydrogel to the subject in order to detect oral disease biomarkers in the subject's mouth and to diagnose the subject with a disease or condition, such as the scanning stage 1104, the diagnosis stage 1106, the monitoring stage 1112, and/or the retention stage 1114. At any of these stages, a hydrogel can be provided to the subject, e.g., via an oral device that is administered to the subject at a dental office, by direct application of the hydrogel to the subject's mouth at a dental office, via an oral device shipped to the subject that the subject can use at a location remote from a dental office (e.g., at the subject's home), via a hydrogel shipped to the subject that the subject can directly apply to the mouth at a location remote from a dental office, etc. Imaging of the oral device and/or hydrogel may be performed at a dental office (e.g., via intraoral or extraoral scanning) or remotely by the subject (e.g., using a mobile device of the subject such as a smartphone with the appropriate imaging capabilities). Alternatively or in combination, at any of these stages, saliva from the subject's mouth can be collected (e.g., at the dental office or at a remote location), and the collected saliva can be applied to a hydrogel. The hydrogel can then be imaged (e.g., via extraoral scanning) to detect oral disease biomarkers. This process can be performed in a dental lab or other facility that is offsite, remote, coupled to, or located in a dental office.

In some embodiments, some or all of the processes described with respect to the workflow 1100 are implemented as computer-readable instructions (e.g., program code) that are configured to be executed by one or more processors of a computing device or system. For example, a computing system that implements the workflow 1100 may include a clinician system implementing software that interfaces with a clinician to provide access to tools for viewing scan data and/or other patient data, inputting diagnoses and/or treatment prescriptions, monitoring treatment progress, and/or accessing reimbursement software. The computing system may include a patient system implementing software that interfaces with the subject to allow the subject to access a digital patient account, set preferences, and/or connect to virtual care systems for remote monitoring (e.g., during the treatment stage 1110 and/or the retention stage 1114). The computing system may include a treatment planning system that generates dental treatment plans and/or dental appliance designs for the subject, e.g., based on a treatment prescription from the clinician system, scan data of the subject's dentition, and/or diagnoses of disease or conditions. The computing system can include a virtual care system that allows for remote treatment monitoring and progress tracking through, e.g., the clinician system and/or the patient system. The computing system may include a reimbursement system that processes insurance claims for oral health procedures (e.g., identifying reimbursement codes related to diagnosis of disease or conditions, arranging reimbursement claims for the diagnosis using the reimbursement code).

II. Dental Appliances and Associated Methods

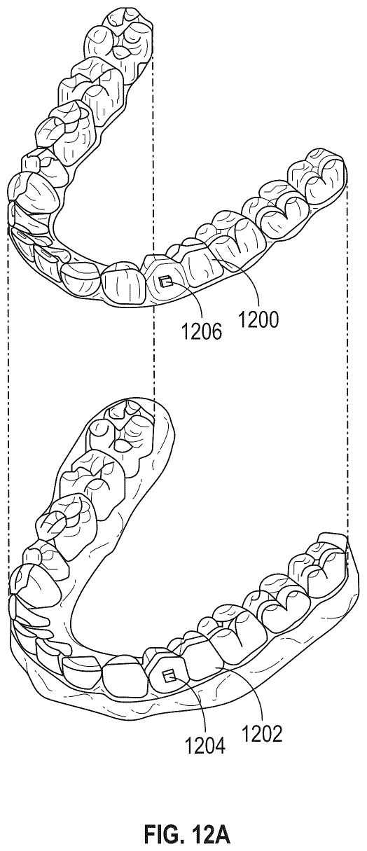

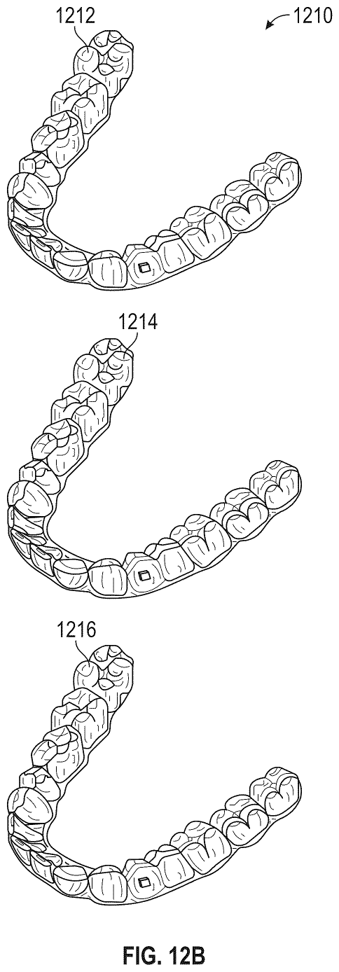

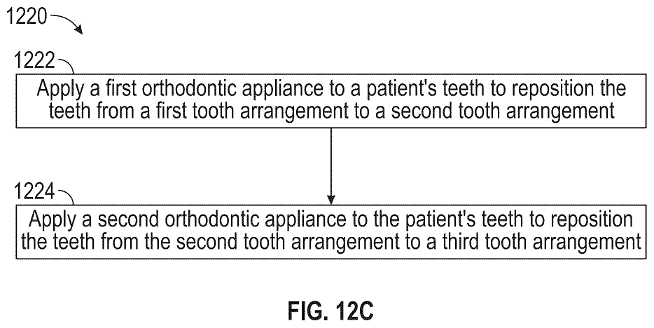

FIG. 12A illustrates a representative example of a tooth repositioning appliance 1200 configured in accordance with embodiments of the present technology. The appliance 1200 can be used with any of the systems, methods, and devices described herein. The appliance 1200 (also referred to herein as an “aligner”) can be worn by a patient in order to achieve an incremental repositioning of individual teeth 1202 in the jaw. The appliance 1200 can include a shell (e.g., a continuous polymeric shell or a segmented shell) having teeth-receiving cavities that receive and resiliently reposition the teeth. The appliance 1200 or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using additive manufacturing techniques, from a digital model of an appliance.

The appliance 1200 can fit over all teeth present in an upper or lower jaw, or less than all of the teeth. The appliance 1200 can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance 1200 can be a generic appliance configured to receive the teeth, but not necessarily shaped to match the topography of the patient's teeth. In some cases, only certain teeth received by the appliance 1200 are repositioned by the appliance 1200 while other teeth can provide a base or anchor region for holding the appliance 1200 in place as it applies force against the tooth or teeth targeted for repositioning. In some cases, some, most, or even all of the teeth can be repositioned at some point during treatment. Teeth that are moved can also serve as a base or anchor for holding the appliance as it is worn by the patient. In preferred embodiments, no wires or other means are provided for holding the appliance 1200 in place over the teeth. In some cases, however, it may be desirable or necessary to provide individual attachments 1204 or other anchoring elements on teeth 1202 with corresponding receptacles 1206 or apertures in the appliance 1200 so that the appliance 1200 can apply a selected force on the tooth. Representative examples of appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 12B illustrates a tooth repositioning system 1210 including a plurality of appliances 1212, 1214, 1216, in accordance with embodiments of the present technology. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 1210 can include a first appliance 1212 corresponding to an initial tooth arrangement, one or more intermediate appliances 1214 corresponding to one or more intermediate arrangements, and a final appliance 1216 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 12C illustrates a method 1220 of orthodontic treatment using a plurality of appliances, in accordance with embodiments of the present technology. The method 1220 can be practiced using any of the appliances or appliance sets described herein. In block 1222, a first orthodontic appliance is applied to a patient's teeth in order to reposition the teeth from a first tooth arrangement to a second tooth arrangement. In block 1224, a second orthodontic appliance is applied to the patient's teeth in order to reposition the teeth from the second tooth arrangement to a third tooth arrangement. The method 1220 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

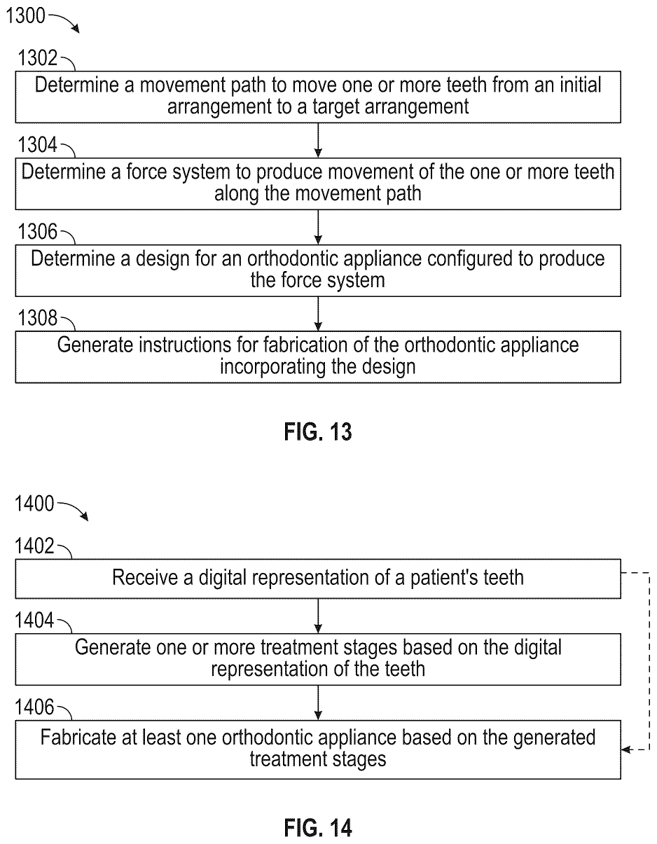

FIG. 13 illustrates a method 1300 for designing an orthodontic appliance, in accordance with embodiments of the present technology. The method 1300 can be applied to any embodiment of the orthodontic appliances described herein. Some or all of the steps of the method 1300 can be performed by any suitable data processing system or device, e.g., one or more processors configured with suitable instructions.

In block 1302, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pre-treatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 1304, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

Determination of the force system can be performed in a variety of ways. For example, in some embodiments, the force system is determined on a patient-by-patient basis, e.g., using patient-specific data. Alternatively or in combination, the force system can be determined based on a generalized model of tooth movement (e.g., based on experimentation, modeling, clinical data, etc.), such that patient-specific data is not necessarily used. In some embodiments, determination of a force system involves calculating specific force values to be applied to one or more teeth to produce a particular movement. Alternatively, determination of a force system can be performed at a high level without calculating specific force values for the teeth. For instance, block 1304 can involve determining a particular type of force to be applied (e.g., extrusive force, intrusive force, translational force, rotational force, tipping force, torquing force, etc.) without calculating the specific magnitude and/or direction of the force.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients can require lower forces to expand the suture than older patients, as the suture has not yet fully formed.

In block 1306, a design for an orthodontic appliance configured to produce the force system is determined. The design can include the appliance geometry, material composition and/or material properties, and can be determined in various ways, such as using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

Optionally, one or more designs can be selected for testing or force modeling. As noted above, a desired tooth movement, as well as a force system required or desired for eliciting the desired tooth movement, can be identified. Using the simulation environment, a candidate design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 1308, instructions for fabrication of the orthodontic appliance incorporating the design are generated. The instructions can be configured to control a fabrication system or device in order to produce the orthodontic appliance with the specified design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Although the above steps show a method 1300 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 1300 may be performed with any suitable fabrication system or device, such as the embodiments described herein. Some of the steps may be optional, e.g., the process of block 1304 can be omitted, such that the orthodontic appliance is designed based on the desired tooth movements and/or determined tooth movement path, rather than based on a force system. Moreover, the order of the steps can be varied as desired.

FIG. 14 illustrates a method 1400 for digitally planning an orthodontic treatment and/or design or fabrication of an appliance, in accordance with embodiments. The method 1400 can be applied to any of the treatment procedures described herein and can be performed by any suitable data processing system.

In block 1402, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 1404, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 1406, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 14, design and/or fabrication of an orthodontic appliance, and perhaps a particular orthodontic treatment, may include use of a representation of the patient's teeth (e.g., including receiving a digital representation of the patient's teeth (block 1402)), followed by design and/or fabrication of an orthodontic appliance based on a representation of the patient's teeth in the arrangement represented by the received representation.

As noted herein, the techniques described herein can be used in combination with dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

The techniques described herein can also be used in combination with attachment placement devices, e.g., appliances used to position prefabricated attachments on a person's teeth in accordance with one or more aspects of a treatment plan. Examples of attachment placement devices (also known as “attachment placement templates” or “attachment fabrication templates”) can be found at least in: U.S. application Ser. No. 17/249,218, entitled “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

The techniques described herein can be used in combination with incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

### III. Overview of Intraoral Scanning Technology

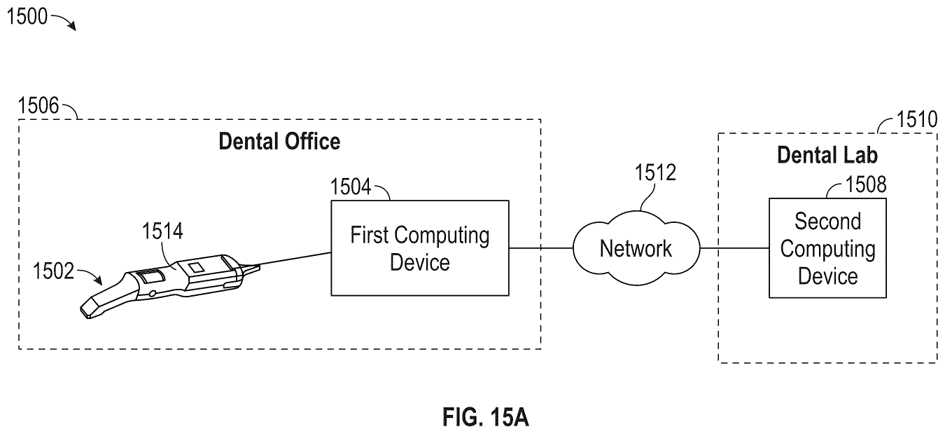

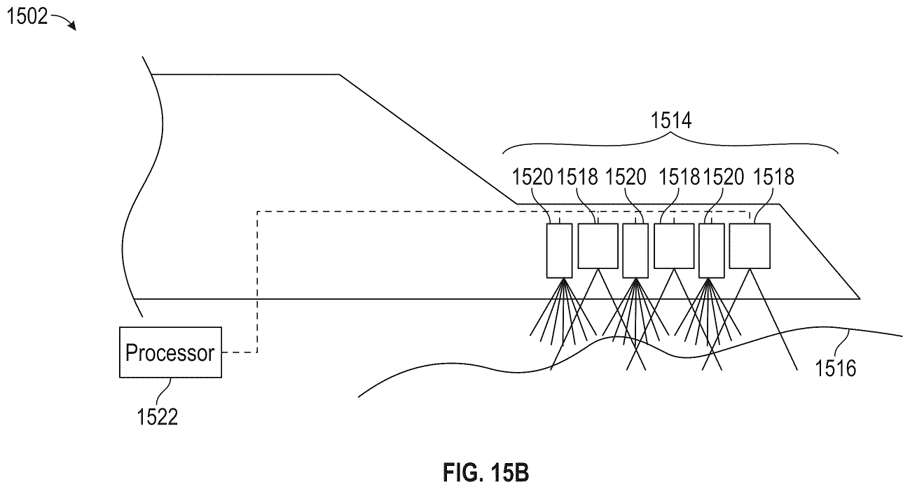

FIG. 15A schematically illustrates a system 1500 for performing intraoral scanning and/or generating 3D digital representations of a patient's intraoral cavity, in accordance with embodiments of the present technology. The system 1500 may be used to generate scan data for any of the methods described herein (e.g., in Section I above). The system 1500 includes an intraoral scanner 1502 (also referred to as a “scanner”) operably coupled to a first computing device 1504. The scanner 1502 and first computing device 1504 can be at a first location, such as a dental office 1506. Optionally, the first computing device 1504 may be operably coupled to another second computing device 1508 at a second location, such as a dental lab 1510. The first computing device 1504 and the second computing device 1508 can be connected to one another via a network 1512, such as a local area network (LAN), a public wide area network (WAN) (e.g., the Internet), a private WAN (e.g., an intranet), or a combination thereof.

The scanner 1502 may be used to generate scan data of one or more intraoral structures of a patient, such as the teeth, gingiva, palate, tongue, cheeks, etc. The scan data can include 3D intraoral scans that provide a digital representation of the surface topography of the intraoral structures. For instance, the 3D intraoral scans can include one or more point clouds, height/depth maps, or any other suitable digital data format depicting the 3D geometry of the intraoral structures. Optionally, the scan data can include other types of digital data, such as color images and/or images obtained at various wavelengths (e.g., near-infrared (NIR) images, infrared images, ultraviolet images, fluorescent images, etc.). In some embodiments, the scanner 1502 alternates between generation of 3D intraoral scans and one or more types of 2D intraoral images (e.g., color images, NIR images, infrared images, ultraviolet images, fluorescent images) during scanning.

In some embodiments, the scanner 1502 includes a probe 1514 (e.g., a handheld wand) that may be inserted at least partially into the intraoral cavity. The probe 1514 can include or be coupled to one or more optical elements for outputting light toward intraoral structures and optically capturing features (e.g., surface topography, color) of the intraoral structures, such as one or more imaging devices (e.g., cameras), light sources (e.g., projectors, lasers), image sensors (e.g., CCD sensors, CMOS sensors), focusing optics (e.g., confocal optics), mirrors, prisms, lenses, beam splitters, polarizers, filters, etc. For instance, for fluorescent imaging, the probe 1514 can include light sources and/or filters for producing the appropriate excitation wavelengths and for obtaining images at the appropriate emission wavelengths. The probe 1514 can include a transparent or translucent window to allow light to pass out of the probe 1514 toward the intraoral structures, and to allow light from the intraoral structures to be received by the probe 1514.

FIG. 15B is a partially schematic illustration of an example scanner 1502 that may be used in the system 1500 of FIG. 15A, in accordance with embodiments of the present technology. The scanner 1502 can be used to obtain scan data of an intraoral surface 1516. In some embodiments, the scanner 1502 includes a probe 1514 at a distal end of the scanner 1502. One or more cameras 1518 are disposed within the probe 1514 (e.g., rigidly fixed within the probe 1514) and arranged within the probe 1514 such that the cameras 1518 receive rays of light from an intraoral cavity in a non-central manner (e.g., the relationship between points in 3D world-coordinate space and corresponding points on the camera sensors of the one or more cameras 1518 is described by a set of camera rays for which there is no single point in space through which all of the camera rays pass).

In some embodiments, the scanner 1502 is configured to perform intraoral scanning using structured light illumination. In such embodiments, one or more structured light projectors 1520 can be disposed within the probe 1514 and can project a pattern of structured light (e.g., a pattern of spots) onto the intraoral surface 1516. Each camera 1518 can be configured to capture a plurality of images that depict at least a portion of the projected pattern of structured light on the intraoral surface 1516. In some embodiments, the structured light projectors 1520 and cameras 1518 are arranged in a closely packed and/or alternating fashion, such that a substantial part of each camera's field of view overlaps the field of view of neighboring cameras 1518, and a substantial part of each projector's field of illumination overlaps the field of illumination of neighboring projectors 1520. The positioning of the projectors 1520 and the cameras 1518 within the probe 1514 can allow the scanner 1502 to have an overall large field of view while maintaining a low profile probe geometry.

The scanner 1502 can further include a processor 1522 configured to generate a 3D model of the intraoral surface 1516 based on images from one or more cameras 1518. In some embodiments, the processor 1522 solves a “correspondence problem,” where a correspondence between pattern elements in the structured light pattern and pattern elements seen by a camera 1518 viewing the pattern is determined. The processor 1522 may compensate for the image distortion specifically introduced by the non-central manner in which one or more cameras 1518 receive rays of light from the intraoral surface 1516 by altering the coordinates of one or more of the structured light pattern elements as seen by one or more cameras 1518 in order to account for the non-central manner in which the one or more cameras 1518 receive rays of light from the intraoral surface 1516.

Referring again to FIG. 15A, other types of scanners 1502 can be used in the system 1500, alternatively or in addition to the embodiment of FIG. 15B. For instance, in some embodiments, the scanner 1502 can be a confocal imaging apparatus including a light source that emits an array of light beams. The light source can be located at a proximal end of the probe 1514, and the probe 1514 can define a light transmission path from the proximal end of the probe 1514 to the distal end of the probe. A set of confocal focusing optics may be positioned along the light transmission path between the proximal end and distal end of the probe 1514. At the distal end, the probe 1514 can include a mirror that directs the array of light beams towards an object outside of the scanner 1502. The light beams reflected off the object can pass back into the probe 1514 and be directed onto an image sensor. In some embodiments, the image sensor detects light intensity at each pixel, which may be used to compute height or depth.

Optionally, the scanner 1502 may include other components, such as a movement sensor for measuring movement and/or pose of the scanner 1502. For example, the movement sensor can be an internal measurement unit (IMU) (e.g., a micro-electromechanical system (MEMS) IMU), which may include one or more accelerometers, gyroscopes, magnetometers, pressure sensors, etc. As another example, the scanner 1502 can include a temperature sensor and temperature control circuitry for measuring and controlling the temperature within the probe 1514, e.g., to reducing fogging of optical elements and/or avoid patient discomfort. In a further example, the scanner 1502 may be used in conjunction with a removable sleeve or other protective device that fits over the probe 1514 to avoid contamination and/or for patient protection. The sleeve may be a single-use component or may be reusable.

Representative examples of intraoral scanners that may be used as the scanner 1502 are described in U.S. Pat. Nos. 11,563,929 and 11,896,461, the disclosures of each of which are incorporated by reference herein in their entirety.

The scanner 1502 can be coupled to the first computing device 1504 via a wired or wireless connection. In some embodiments, the scanner 1502 is wirelessly connected to the first computing device 1504 via a direct wireless connection. In some embodiments, the scanner 1502 is wirelessly connected to the first computing device 1504 via a wireless network, such as a Wi-Fi network, Bluetooth network, a Zigbee network, or other wireless network. For example, the first computing device 1504 may be physically connected to one or more wireless access points and/or wireless routers (e.g., Wi-Fi access points/routers), and the scanner 1502 may include a wireless module (e.g., a Wi-Fi module) for joining the wireless network via the wireless access point and/or router.

The scan data obtained by the scanner 1502 may be transmitted to the first computing device 1504, and the first computing device 1504 may store the scan data in a data store. The data store may include local data stores and/or remote data stores. The first computing device 1504 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components. The first computing device 1504 can also include software components for monitoring and controlling the scanner 1502, receiving and processing the scan data, and/or other functionality relevant to an intraoral scanning procedure.

In some embodiments, a user (e.g., a patient, a clinician, technician, or other practitioner) performs intraoral scanning of a patient in connection with a dental procedure. By way of non-limiting example, dental procedures may be broadly divided into prosthodontic (restorative) and orthodontic procedures, and then further subdivided into specific forms of these procedures. Additionally, dental procedures may include identification and treatment of periodontal disease, sleep apnea, and intraoral conditions. The term prosthodontic procedure may refer to any procedure involving the oral cavity and directed to the design, manufacture, or installation of a dental prosthesis at a dental site within the oral cavity, or a real or virtual model thereof, or directed to the design and preparation of the dental site to receive such a prosthesis. A prosthesis may include any restoration such as crowns, veneers, inlays, onlays, implants and bridges, for example, and any other artificial partial or complete denture. The term orthodontic procedure may refer to any procedure involving the intraoral cavity and directed to the design, manufacture, or installation of orthodontic elements at a dental site within the intraoral cavity, or a real or virtual model thereof, or directed to the design and preparation of the dental site to receive such orthodontic elements. These elements may be appliances including but not limited to brackets and wires, retainers, aligners, palatal expanders, attachment placement templates, mouth guards, oral sleep apnea devices, or other dental appliances.

In some embodiments, intraoral scanning is performed on a patient's intraoral cavity during a visitation of the dental office 1506. The intraoral scanning may be performed, for example, as part of a semi-annual or annual dental health checkup. The intraoral scanning may also be performed before, during and/or after one or more dental treatments, such as orthodontic treatment and/or prosthodontic treatment. The intraoral scanning may be a full or partial scan of the upper and/or lower dental arches, and may be performed in order to gather information for performing dental and/or periodontal diagnostics, to generate a treatment plan, to determine progress of a treatment plan, and/or for other purposes.

During an intraoral scanning procedure, the user may apply the scanner 1502 to one or more locations within the intraoral cavity of the patient. The scanning may be divided into one or more segments. As an example, the segments may include a lower dental arch of the patient (e.g., the complete lower dental arch or a portion thereof), an upper dental arch of the patient (e.g., the complete upper dental arch or a portion thereof), and/or patient bite (e.g., scanning performed with closure of the patient's mouth with the scan being directed towards an interface area of the patient's upper and lower teeth). Via such scanner application, the scanner 1502 may provide scan data to the first computing device 1504. The scan data may be provided in the form of intraoral scan data sets, each of which may include 3D intraoral scans (e.g., point clouds, height/depth maps) and/or 2D intraoral images (e.g., color images, NIR images, infrared images, ultraviolet images).

The first computing device 1504 can include one or more software components configured to process the scan data into a 3D digital representation of the patient's intraoral structures. For example, the first computing device 1504 can implement an intraoral scan application that registers and stitches together two or more intraoral scans from the scan data to generate a growing 3D surface. In some embodiments, performing registration includes capturing 3D data of various points of a surface in multiple scans, and registering the scans by computing transformations between the scans (e.g., based on overlapping points depicted in the scans). One or more 3D surfaces may be generated based on the registered and stitched together intraoral scans during the intraoral scanning. The one or more 3D surfaces may be output to a graphical user interface (GUI) on a display of the first computing device 1504 so that the user can view the scan progress thus far. As each new intraoral scan is captured and registered to previous intraoral scans and/or to the generated 3D surface(s), the 3D surface(s) may be updated, and the updated 3D surface(s) may be output to the display. The user interface showing the 3D surface(s) may be periodically or continuously updated to show scanning progress in real time or near-real time.

When a scan session or a portion of a scan session associated with a particular scanning segment (e.g., upper dental arch, lower dental arch, bite) is complete (e.g., all scans for the site of interest have been captured), the intraoral scan application may generate a 3D digital representation of the scanned segment (e.g., a virtual 3D model). The 3D digital representation may be a set of 3D points and their connections with each other (e.g., a mesh). To generate the 3D digital representation, the intraoral scan application may register and stitch together the intraoral scans generated from the intraoral scan session that are associated with a particular scanning segment. The registration performed at this stage may be more accurate than the registration performed during the capturing of the intraoral scans, and may take more time to complete than the registration performed during the capturing of the intraoral scans. In some embodiments, performing scan registration includes capturing 3D data of various points of a surface in multiple scans, and registering the scans by computing transformations between the scans. The 3D data may be projected into a 3D space of the 3D digital representation to form a portion of the 3D digital representation. The intraoral scans may be integrated into a common reference frame by applying appropriate transformations to points of each registered scan and projecting each scan into the 3D space.

In some embodiments, registration is performed for adjacent or overlapping intraoral scans (e.g., each successive frame of an intraoral video). Registration algorithms may be carried out to register two adjacent or overlapping intraoral scans and/or to register an intraoral scan with a 3D digital representation, which can involve determination of the transformations which align one scan with the other scan and/or with the 3D digital representation. Registration may involve identifying multiple points in each scan (e.g., point clouds) of a scan pair (or of a scan and the 3D digital representation), surface fitting to the points, and using local searches around points to match points of the two scans (or of the scan and the 3D digital representation). For example, the intraoral scan application may match points of one scan with the closest points interpolated on the surface of another scan, and iteratively minimize the distance between matched points. Other registration techniques known to those of skill in the art may also be used. Examples of registration techniques include, for example, iterative closest point (ICP) algorithms.

The intraoral scan application may repeat registration for all intraoral scans of a sequence of intraoral scans to obtain transformations for each intraoral scan, to register each intraoral scan with previous intraoral scan(s) and/or with a common reference frame (e.g., with the 3D digital representation). Intraoral scan application may integrate intraoral scans into a single 3D digital representation by applying the appropriate determined transformations to each of the intraoral scans. Each transformation may include rotations about one to three axes and/or translations along one to three axes.

The intraoral scan application may generate one or more 3D digital representations from intraoral scans, and may display the 3D digital representation(s) to the user via a GUI on the display. The 3D digital representation(s) can then be checked visually by the user. The user can virtually manipulate the 3D digital representation(s) via the user interface with respect to up to six degrees of freedom (e.g., translated and/or rotated with respect to one or more of three mutually orthogonal axes) using suitable user controls (e.g., hardware and/or software controls) to enable viewing of the 3D digital representation(s) from any desired direction.

Optionally, the scan data generated by the scanner 1502 and/or the 3D digital representation(s) generated by the intraoral scan application may be transmitted from the first computing device 1504 to the second computing device 1508 via the network 1512. The second computing device 1508 can be coupled to a data store for storing the scan data and/or the 3D digital representations, which may include local data stores and/or remote data stores. The second computing device 1508 can be a personal computer, workstation, laptop, tablet, smartphone, etc., that includes one or more processors, memory, secondary storage devices, input devices (e.g., a keyboard, mouse, tablet, touchscreen, microphone, camera), output devices (e.g., display, printer, touchscreen, speakers), and/or other suitable hardware components.

In some embodiments, the second computing device 1508 includes one or more software components configured to perform dental and/or periodontal diagnostics, generate a treatment plan, to determine progress of a treatment plan, and/or for other purposes relevant to a dental procedure, based on the scan data and/or the 3D digital representation(s). For example, a 3D digital representation of a patient's intraoral cavity may be used to design a dental prosthesis for a prosthodontic procedure, such as one or more crowns, veneers, inlays, onlays, implants, bridges, etc. As another example, a 3D digital representation of a patient's intraoral cavity may be used to design a dental appliance for an orthodontic procedure, such as one more aligners, retainers, palatal expanders, etc. In a further example, a 3D digital representation of a patient's intraoral cavity may be used to diagnose a patient with periodontal disease, sleep apnea, and/or other intraoral conditions.

The system 1500 can be configured in many different ways. For example, any of the components of the system 1500 shown as distinct elements in FIG. 15A can be combined into a single device, and/or any of the components of the system 1500 shown as a single element in FIG. 15A can be divided into a plurality of discrete devices. Moreover, the locations of the components of the system 1500 can be varied as desired, e.g., any of the components shown in FIG. 15A can be located at the dental office 1506, the dental lab 1510, or at one or more other locations, such as a server farm that provides a cloud computing service, a facility of a manufacturer of the scanner 1502, a facility of a manufacturer of dental appliances and/or dental prosthetics, etc. Additionally, any of the operations that are described as being performed by a particular component of the system 1500 can alternatively or additionally be performed by any other component of the system 1500, e.g., the operations of the first computing device 1504 may alternatively or additionally be performed by the second computing device 1508 and/or by another computing device (e.g., a remote server), and vice-versa.

The system 1500 may include additional components not illustrated in FIG. 15A. For instance, although FIG. 15A depicts a single scanner 1502, the system 1500 can optionally include multiple scanners 1502, which may be at the same location (e.g., the same dental office 1506) or at different locations (e.g., different dental offices 1506). Similarly, although FIG. 15A depicts a single dental office 1506 and a single dental lab 1510, the system 1500 may include multiple dental offices 1506, multiple dental labs 1510, and/or other facilities including respective computing devices that are communicably coupled to each other via one or more networks 1512 in any suitable arrangement.

### EXAMPLES

The following examples are included to further describe some aspects of the present technology, and should not be used to limit the scope of the technology.

Example 1. A system for detecting a disease biomarker in a subject, the device comprising:
an oral device configured to be positioned within a subject's mouth;
a hydrogel carried by the oral device, wherein the hydrogel comprises a polymer and a capture moiety coupled to the polymer, and wherein the capture moiety is configured to bind to a disease biomarker present in the subject's mouth; and
a fluorescent moiety configured to label the disease biomarker bound by the capture moiety.

Example 2. The system of Example 1, wherein the oral device comprises a dental appliance.

Example 3. The system of Example 2, wherein the dental appliance is an aligner, a retainer, or a palatal expander.

Example 4. The system of Example 2 or 3, wherein the dental appliance comprises a shell having a plurality of teeth-receiving cavities, and wherein the hydrogel is coated onto a surface of the shell.

Example 5. The system of Example 4, wherein the surface comprises a buccal surface, a lingual surface, an occlusal surface, an interior surface, an exterior surface, or a combination thereof.

Example 6. The system of Example 1, wherein the oral device comprises a lollipop-type device having an intraoral portion coupled to a support shaft.

Example 7. The system of Example 6, wherein the hydrogel is coated on the intraoral portion.

Example 8. The system of Example 6 or 7, wherein the intraoral portion is composed of the hydrogel.

Example 9. The system of any one of Examples 1 to 8, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 10. The system of any one of Examples 1 to 9, wherein the capture moiety is covalently coupled to the polymer.

Example 11. The system of any one of Examples 1 to 10, wherein the capture moiety comprises an antibody, an aptamer, a peptide, a polysaccharide, or a small molecule.

Example 12. The system of any one of Examples 1 to 11, wherein the capture moiety is configured to specifically bind to the disease biomarker.

Example 13. The system of any one of Examples 1 to 11, wherein the capture moiety is configured to bind to a plurality of species present in the subject's mouth.

Example 14. The system of any one of Examples 1 to 13, wherein the fluorescent moiety comprises an organic dye, a polymer dye, a fluorescent protein, or a quantum dot.

Example 15. The system of any one of Examples 1 to 14, wherein the fluorescent moiety is configured to bind to the disease biomarker.

Example 16. The system of any one of Examples 1 to 15, wherein the disease biomarker is present in saliva.

Example 17. The system of any one of Examples 1 to 16, wherein the disease biomarker is a protein, a peptide, a small molecule, a nucleic acid, a polysaccharide, or a lipid.

Example 18. The system of any one of Examples 1 to 17, wherein the disease biomarker is a biomarker for oral cancer.

Example 19. The system of any one of Examples 1 to 17, wherein the disease biomarker is a biomarker for caries, orthodontic bone remodeling, or periodontitis.

Example 20. The system of any one of Examples 1 to 17, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 21. The system of any one of Examples 1 to 20, further comprising an imaging device configured to obtain image data of the labeled disease biomarker.

Example 22. The system of Example 21, wherein the imaging device is an intraoral scanner.

Example 23. The system of Example 21, wherein the imaging device is a mobile device.

Example 24. A method for detecting a disease biomarker in a subject, the device comprising:
positioning an oral device within a subject's mouth, wherein the oral device comprises a hydrogel including a polymer and a capture moiety coupled to the polymer;
binding a disease biomarker present in the subject's mouth with the capture moiety;
labeling the disease biomarker bound by the capture moiety with a fluorescent moiety; and
obtaining image data of the oral device including the hydrogel with the labeled disease biomarker.

Example 25. The method of Example 24, wherein the oral device comprises a dental appliance.

Example 26. The method of Example 25, wherein the dental appliance is an aligner, a retainer, or a palatal expander.

Example 27. The method of Example 25 or 26, wherein the dental appliance comprises a shell having a plurality of teeth-receiving cavities, and wherein the hydrogel is coated onto a surface of the shell.

Example 28. The method of Example 27, wherein the surface comprises a buccal surface, a lingual surface, an occlusal surface, an interior surface, an exterior surface, or a combination thereof.

Example 29. The method of Example 24, wherein the oral device comprises a lollipop-type device having an intraoral portion coupled to a support shaft.

Example 30. The method of Example 29, wherein the hydrogel is coated on the intraoral portion.

Example 31. The method of Example 29 or 30, wherein the intraoral portion is composed of the hydrogel.

Example 32. The method of any one of Examples 24 to 31, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 33. The method of any one of Examples 24 to 32, wherein the capture moiety is covalently coupled to the polymer.

Example 34. The method of any one of Examples 24 to 33, wherein the capture moiety comprises an antibody, an aptamer, a peptide, a polysaccharide, or a small molecule.

Example 35. The method of any one of Examples 24 to 34, wherein the capture moiety is configured to specifically bind to the disease biomarker.

Example 36. The method of any one of Examples 24 to 34, wherein the capture moiety is configured to bind to a plurality of species present in the subject's mouth.

Example 37. The method of any one of Examples 24 to 36, wherein the fluorescent moiety comprises an organic dye, a polymer dye, a fluorescent protein, or a quantum dot.

Example 38. The method of any one of Examples 24 to 37, wherein the fluorescent moiety is configured to bind to the disease biomarker.

Example 39. The method of any one of Examples 24 to 38, wherein the disease biomarker is present in saliva.

Example 40. The method of any one of Examples 24 to 39, wherein the disease biomarker is a protein, a peptide, a small molecule, a nucleic acid, a polysaccharide, or a lipid.

Example 41. The method of any one of Examples 24 to 40, wherein the disease biomarker is a biomarker for oral cancer.

Example 42. The method of any one of Examples 24 to 40, wherein the disease biomarker is a biomarker for caries, orthodontic bone remodeling, or periodontitis.

Example 43. The method of any one of Examples 24 to 40, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 44. The method of any one of Examples 24 to 43, wherein the image data is obtained by scanning the oral device using an intraoral scanner.

Example 45. The method of any one of Examples 24 to 43, wherein the image data is obtained using a mobile device.

Example 46. The method of any one of Examples 24 to 45, further comprising removing the oral device from the subject's mouth before labeling the disease biomarker and obtaining the image data.

Example 47. The method of any one of Examples 24 to 46, further comprising detecting whether the labeled biomarker is present in the image data.

Example 48. The method of Example 47, further comprising diagnosing the subject with a disease based on the presence of the labeled biomarker in the image data.

Example 49. The method of Example 48, further comprising comparing the image data to additional data of the subject, wherein the diagnosis is based on the comparison.

Example 50. The method of Example 49, wherein the additional data comprises previous image data of an oral device comprising a hydrogel with a labeled disease biomarker.

Example 51. The method of Example 49 or 50, wherein the additional data comprises one or more of radiographic data, intraoral scan data, photographs, or videos.

Example 52. The method of any one of Examples 47 to 51, further comprising:
identifying a location of the labeled biomarker relative to the oral device, and
determining a disease site in the subject's mouth based on the identified location.

Example 53. A system for detecting a disease biomarker in a subject, the device comprising:
an oral device configured to be positioned within a subject's mouth; and
a hydrogel carried by the oral device, the hydrogel comprising a polymer and a fluorescent moiety,
wherein when the oral device is positioned within the subject's mouth, the hydrogel is configured to release the fluorescent moiety into the subject's mouth, and
wherein the fluorescent moiety is configured to label a disease biomarker present in the subject's mouth.

Example 54. The system of Example 53, wherein the oral device comprises a dental appliance.

Example 55. The system of Example 54, wherein the dental appliance is an aligner, a retainer, or a palatal expander.

Example 56. The system of Example 54 or 55, wherein the dental appliance comprises a shell having a plurality of teeth-receiving cavities, and wherein the hydrogel is coated onto a surface of the shell.

Example 57. The system of Example 56, wherein the surface comprises a buccal surface, a lingual surface, an occlusal surface, an interior surface, an exterior surface, or a combination thereof.

Example 58. The system of Example 53, wherein the oral device comprises a lollipop-type device having an intraoral portion coupled to a support shaft.

Example 59. The system of Example 58, wherein the hydrogel is coated on the intraoral portion.

Example 60. The system of Example 58 or 59, wherein the intraoral portion is composed of the hydrogel.

Example 61. The system of any one of Examples 53 to 60, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 62. The system of any one of Examples 53 to 61, wherein the fluorescent moiety comprises an organic dye, a polymer dye, a fluorescent protein, or a quantum dot.

Example 63. The system of any one of Examples 53 to 62, wherein the fluorescent moiety comprises an antibody, an aptamer, a peptide, a polysaccharide, or a small molecule that is configured to bind to the disease biomarker.

Example 64. The system of any one of Examples 53 to 63, wherein the disease biomarker is present in saliva.

Example 65. The system of any one of Examples 53 to 64, wherein the disease biomarker is a protein, a peptide, a small molecule, a nucleic acid, a polysaccharide, or a lipid.

Example 66. The system of any one of Examples 53 to 65, wherein the disease biomarker is a biomarker for oral cancer.

Example 67. The system of any one of Examples 53 to 65, wherein the disease biomarker is a biomarker for caries, orthodontic bone remodeling, or periodontitis.

Example 68. The system of any one of Examples 53 to 65, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 69. The system of any one of Examples 53 to 68, further comprising an imaging device configured to obtain image data of the labeled disease biomarker.

Example 70. The system of Example 69, wherein the imaging device is an intraoral scanner.

Example 71. The system of Example 69, wherein the imaging device is a mobile device.

Example 72. A method for detecting a disease biomarker in a subject, the device comprising:
positioning an oral device within a subject's mouth, wherein the oral device comprises a hydrogel including a polymer and a fluorescent moiety;
releasing the fluorescent moiety into the subject's mouth;
labeling a disease biomarker present in the subject's mouth with the fluorescent moiety; and
obtaining image data of the labeled disease biomarker in the subject's mouth.

Example 73. The method of Example 72, wherein the oral device comprises a dental appliance.

Example 74. The method of Example 73, wherein the dental appliance is an aligner, a retainer, or a palatal expander.

Example 75. The method of Example 73 or 74, wherein the dental appliance comprises a shell having a plurality of teeth-receiving cavities, and wherein the hydrogel is coated onto a surface of the shell.

Example 76. The method of Example 75, wherein the surface comprises a buccal surface, a lingual surface, an occlusal surface, an interior surface, an exterior surface, or a combination thereof.

Example 77. The method of Example 72, wherein the oral device comprises a lollipop-type device having an intraoral portion coupled to a support shaft.

Example 78. The method of Example 77, wherein the hydrogel is coated on the intraoral portion.

Example 79. The method of Example 77 or 78, wherein the intraoral portion is composed of the hydrogel.

Example 80. The method of any one of Examples 72 to 79, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 81. The method of any one of Examples 72 to 80, wherein the fluorescent moiety comprises an organic dye, a polymer dye, a fluorescent protein, or a quantum dot.

Example 82. The method of any one of Examples 72 to 81, wherein the fluorescent moiety comprises an antibody, an aptamer, a peptide, a polysaccharide, or a small molecule that is configured to bind to the disease biomarker.

Example 83. The method of any one of Examples 72 to 82, wherein the disease biomarker is present in saliva.

Example 84. The method of any one of Examples 72 to 83, wherein the disease biomarker is a protein, a peptide, a small molecule, a nucleic acid, a polysaccharide, or a lipid.

Example 85. The method of any one of Examples 72 to 84, wherein the disease biomarker is a biomarker for oral cancer.

Example 86. The method of any one of Examples 72 to 84, wherein the disease biomarker is a biomarker for caries, orthodontic bone remodeling, or periodontitis.

Example 87. The method of any one of Examples 72 to 84, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 88. The method of any one of Examples 72 to 87, wherein the image data is obtained by scanning the subject's mouth using an intraoral scanner.

Example 89. The method of any one of Examples 72 to 87, wherein the image data is obtained using a mobile device.

Example 90. The method of any one of Examples 72 to 89, further comprising removing the oral device from the subject's mouth before obtaining the image data.

Example 91. The method of any one of Examples 72 to 90, further comprising detecting whether the labeled biomarker is present in the image data.

Example 92. The method of Example 91, further comprising diagnosing the subject with a disease based on the presence of the labeled biomarker in the image data.

Example 93. The method of Example 92, further comprising comparing the image data to additional data of the subject, wherein the diagnosis is based on the comparison.

Example 94. The method of Example 93, wherein the additional data comprises previous image data of a labeled disease biomarker in the subject's mouth.

Example 95. The method of Example 93 or 94, wherein the additional data comprises one or more of radiographic data, intraoral scan data, photographs, or videos.

Example 96. The method of any one of Examples 91 to 95, further comprising:
identifying a location of the labeled biomarker in the subject's mouth, and
determining a disease site in the subject's mouth based on the identified location.

Example 97. A system for detecting a disease biomarker in a subject, the device comprising:
an oral device configured to be positioned within a subject's mouth; and
a hydrogel carried by the oral device, the hydrogel comprising a polymer and a fluorescent moiety,
wherein the fluorescent moiety is in an inactive state before the oral device is positioned in the subject's mouth, and
wherein when the oral device is positioned within the subject's mouth, the fluorescent moiety is configured to interact with a disease biomarker in the subject's mouth, and the interaction causes the fluorescent moiety to be converted into an active state.

Example 98. The system of Example 97, wherein the oral device comprises a dental appliance.

Example 99. The system of Example 98, wherein the dental appliance is an aligner, a retainer, or a palatal expander.

Example 100. The system of Example 98 or 99, wherein the dental appliance comprises a shell having a plurality of teeth-receiving cavities, and wherein the hydrogel is coated onto a surface of the shell.

Example 101. The system of Example 100, wherein the surface comprises a buccal surface, a lingual surface, an occlusal surface, an interior surface, an exterior surface, or a combination thereof.

Example 102. The system of Example 97, wherein the oral device comprises a lollipop-type device having an intraoral portion coupled to a support shaft.

Example 103. The system of Example 102, wherein the hydrogel is coated on the intraoral portion.

Example 104. The system of Example 102 or 103, wherein the intraoral portion is composed of the hydrogel.

Example 105. The system of any one of Examples 97 to 104, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 106. The system of any one of Examples 97 to 105, wherein the fluorescent moiety comprises an organic dye, a polymer dye, a fluorescent protein, or a quantum dot.

Example 107. The system of any one of Examples 97 to 106, wherein the fluorescent moiety comprises an antibody, an aptamer, a peptide, a polysaccharide, or a small molecule that is configured to bind to the disease biomarker.

Example 108. The system of any one of Examples 97 to 107, wherein the fluorescent moiety is converted into the active state via one or more of the following: an enzymatic reaction, a conformational change, or removal of a fluorescence quencher.

Example 109. The system of any one of Examples 97 to 108, wherein the disease biomarker is present in saliva.

Example 110. The system of any one of Examples 97 to 109, wherein the disease biomarker is a protein, a peptide, a small molecule, a nucleic acid, a polysaccharide, or a lipid.

Example 111. The system of any one of Examples 97 to 110, wherein the disease biomarker is a biomarker for oral cancer.

Example 112. The system of any one of Examples 97 to 110, wherein the disease biomarker is a biomarker for caries, orthodontic bone remodeling, or periodontitis.

Example 113. The system of any one of Examples 97 to 110, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 114. The system of any one of Examples 97 to 113, further comprising an imaging device configured to obtain image data of the fluorescent moiety in the active state.

Example 115. The system of Example 114, wherein the imaging device is an intraoral scanner.

Example 116. The system of Example 114, wherein the imaging device is a mobile device.

Example 117. A method for detecting a disease biomarker in a subject, the device comprising:
positioning an oral device within a subject's mouth, wherein the oral device comprises a hydrogel including a polymer and a fluorescent moiety;
converting the fluorescent moiety from an inactive state to an active state via an interaction between the fluorescent moiety and a disease biomarker in the subject's mouth; and
obtaining image data of the oral device including the hydrogel with the fluorescent moiety.

Example 118. The method of Example 117, wherein the oral device comprises a dental appliance.

Example 119. The method of Example 118, wherein the dental appliance is an aligner, a retainer, or a palatal expander.

Example 120. The method of Example 118 or 119, wherein the dental appliance comprises a shell having a plurality of teeth-receiving cavities, and wherein the hydrogel is coated onto a surface of the shell.

Example 121. The method of Example 120, wherein the surface comprises a buccal surface, a lingual surface, an occlusal surface, an interior surface, an exterior surface, or a combination thereof.

Example 122. The method of Example 117, wherein the oral device comprises a lollipop-type device having an intraoral portion coupled to a support shaft.

Example 123. The method of Example 122, wherein the hydrogel is coated on the intraoral portion.

Example 124. The method of Example 122 or 123, wherein the intraoral portion is composed of the hydrogel.

Example 125. The method of any one of Examples 117 to 124, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

Example 126. The method of any one of Examples 117 to 125, wherein the fluorescent moiety comprises an organic dye, a polymer dye, a fluorescent protein, or a quantum dot.

Example 127. The method of any one of Examples 117 to 126, wherein the fluorescent moiety comprises an antibody, an aptamer, a peptide, a polysaccharide, or a small molecule that is configured to bind to the disease biomarker.

Example 128. The method of any one of Examples 117 to 127, wherein the fluorescent moiety is converted into the active state via one or more of the following: an enzymatic reaction, a conformational change, or removal of a fluorescence quencher.

Example 129. The method of any one of Examples 117 to 128, wherein the disease biomarker is present in saliva.

Example 130. The method of any one of Examples 117 to 129, wherein the disease biomarker is a protein, a peptide, a small molecule, a nucleic acid, a polysaccharide, or a lipid.

Example 131. The method of any one of Examples 117 to 130, wherein the disease biomarker is a biomarker for oral cancer.

Example 132. The method of any one of Examples 117 to 130, wherein the disease biomarker is a biomarker for caries, orthodontic bone remodeling, or periodontitis.

Example 133. The method of any one of Examples 117 to 130, wherein the disease biomarker is a biomarker for Alzheimer's disease, asthma, cardiovascular disease, chronic obstructive pulmonary disease (COPD), cystic fibrosis, diabetes, Epstein-Barr virus (EBV), gastroesophageal reflux disease (GERD), hepatitis, human immunodeficiency virus (HIV), human papillomavirus (HPV), Huntington's disease, oral candidiasis, osteoporosis, Parkinson's disease, sickle cell anemia, Sjogren's syndrome, or substance abuse.

Example 134. The method of any one of Examples 117 to 133, wherein the image data is obtained by scanning the oral device using an intraoral scanner.

Example 135. The method of any one of Examples 117 to 133, wherein the image data is obtained using a mobile device.

Example 136. The method of any one of Examples 117 to 135, further comprising removing the oral device from the subject's mouth before obtaining the image data.

Example 137. The method of any one of Examples 117 to 135, wherein the image data is obtained while the oral device is in the subject's mouth.

Example 138. The method of any one of Examples 117 to 137, further comprising detecting whether the fluorescent moiety is in the active state in the image data.

Example 139. The method of Example 138, further comprising diagnosing the subject with a disease based on the active state of the fluorescent moiety in the image data.

Example 140. The method of Example 139, further comprising comparing the image data to additional data of the subject, wherein the diagnosis is based on the comparison.

Example 141. The method of Example 140, wherein the additional data comprises previous image data of an oral device including a hydrogel with a fluorescent moiety.

Example 142. The method of Example 140 or 141, wherein the additional data comprises one or more of radiographic data, intraoral scan data, photographs, or videos.

Example 143. The method of any one of Examples 138 to 142, further comprising:
identifying a location of the fluorescent moiety in the active state relative to the oral device, and
determining a disease site in the subject's mouth based on the identified location.

### Conclusion

Although many of the embodiments are described above with respect to systems, devices, and methods for detection of oral disease biomarkers, the technology is applicable to other applications and/or other approaches, such as detection of disease biomarkers at other anatomical locations. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-15B.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A system for detecting a disease biomarker in a subject, the system comprising:
an oral device configured to be positioned within a subject's mouth;
a hydrogel carried by the oral device, wherein the hydrogel comprises a polymer and a capture moiety coupled to the polymer, and wherein the capture moiety is configured to bind to a disease biomarker present in a physiological fluid in the subject's mouth, and wherein the disease biomarker is a biomarker for oral cancer;
a fluorescent moiety configured to label the disease biomarker bound by the capture moiety;
an imaging device configured to obtain image data of the oral device and the hydrogel; and
one or more processors configured to perform operations comprising:
detecting whether the labeled disease biomarker is present in the image data, and
determining a diagnosis for the subject based on whether the labeled disease biomarker is present in the image data.

2. The system of claim 1, wherein the oral device comprises a dental appliance.

3. The system of claim 2, wherein the dental appliance is an aligner, a retainer, or a palatal expander.

4. The system of claim 2, wherein the dental appliance comprises a shell having a plurality of teeth-receiving cavities, and wherein the hydrogel is coated onto a surface of the shell.

5. The system of claim 1, wherein the oral device comprises a lollipop-type device having an intraoral portion coupled to a support shaft.

6. The system of claim 1, wherein the polymer comprises agarose, alginate, cellulose, chitosan, collagen, dextran, fibrin, gelatin, guar gum, hyaluronic acid, keratin, pectin, silk, starch, poly(2-hydroxyethyl methacrylate), poly(acrylic acid), poly(caprolactone), poly(ethylene glycol), poly(glycolide), poly(lactic-co-glycolic acid), poly(lactide), poly(methacrylic acid), poly(N-isopropylacrylamide), poly(vinyl alcohol), polyacrylamide, polystyrene, polyurethane, polyvinylpyrrolidone, or a derivative or a combination thereof.

7. The system of claim 1, wherein the capture moiety is covalently coupled to the polymer.

8. The system of claim 1, wherein the capture moiety is configured to specifically bind to the disease biomarker.

9. The system of claim 1, wherein the capture moiety is configured to bind to a plurality of species present in the subject's mouth.

10. The system of claim 1, wherein the fluorescent moiety is configured to bind to the disease biomarker.

11. The system of claim 1, wherein the disease biomarker is present in saliva.

12. (canceled)

13. (canceled)

14. The system of claim 1, wherein the imaging device is an intraoral scanner.

15. A method for detecting a disease biomarker in a subject, the method comprising:
positioning an oral device within a subject's mouth, wherein the oral device comprises a hydrogel including a polymer and a capture moiety coupled to the polymer;
binding a disease biomarker present in a physiological fluid in the subject's mouth with the capture moiety, wherein the disease biomarker is a biomarker for oral cancer;
labeling the disease biomarker bound by the capture moiety with a fluorescent moiety;
obtaining image data of the oral device including the hydrogel with the labeled disease biomarker using an imaging device;
detecting, via one or more processors, whether the labeled disease biomarker is present in the image data; and
determining, via the one or more processors, a diagnosis for the subject based on whether the labeled disease biomarker is present in the image data.

16. The method of claim 15, wherein the oral device comprises a dental appliance.

17. The method of claim 16, wherein the dental appliance is an aligner, a retainer, or a palatal expander.

18. The method of claim 16, wherein the dental appliance comprises a shell having a plurality of teeth-receiving cavities, and wherein the hydrogel is coated onto a surface of the shell.

19. The method of claim 15, wherein the oral device comprises a lollipop-type device having an intraoral portion coupled to a support shaft.

20-28. (canceled)

29. The method of claim 15, further comprising removing the oral device from the subject's mouth before labeling the disease biomarker and obtaining the image data.

30. (canceled)

31. The system of claim 1, wherein the imaging device is a mobile device.

32. The system of claim 1, wherein the image data comprises a plurality of 2D images.

33. The system of claim 32, wherein the plurality of 2D images comprise images obtained using different imaging modalities.

34. The system of claim 1, wherein the operations further comprise identifying a location of a fluorescent signal corresponding to the fluorescent moiety in the image data.

35. The system of claim 34, wherein the operations further comprise:
generating a digital representation of a geometry of the oral device, based on the image data, and
determining a spatial distribution of the fluorescent signal with respect to the geometry of the oral device.

36. The system of claim 34, wherein determining the diagnosis comprises determining a location of the oral cancer in the subject's mouth, based on the identified location of the fluorescent signal.

37. The system of claim 36, wherein determining the diagnosis comprises comparing the determined location of the oral cancer to a location identified from image data obtained using a different imaging modality.

38. The system of claim 1, wherein determining the diagnosis comprises comparing the image data to previous image data.

39. The system of claim 38, wherein the previous image data is obtained at a previous time point.

40. The system of claim 38, wherein the previous image data depicts a second oral device that was previously positioned within the subject's mouth.

41. The system of claim 40, wherein the second oral device includes the hydrogel comprising the polymer and the capture moiety coupled to the polymer.

42. The system of claim 38, wherein the comparing comprises identifying a change in one or more of a presence, amount, or location of the labeled disease biomarker over time.
